(19) United States
(12) Patent Application Publication
(10) Pub. No.: US 2015/0083121 A1
(43) Pub. Date: Mar. 26, 2015

(54) PORTABLE LIFE SUPPORT APPARATUS

(71) Applicants: Joseph Fisher, Thornhill (CA); Bryan Kowalchuk, Oakville (CA); Bryan Drew Miller, Richmond Hill (CA); Kevin Ramkhelawan, Oakville (CA); Kevin Kowalchuk, Oakville (CA); Cliff Ansel, Thornhill (CA); David Dunn, Toronto (CA); Ludwik Fredorko, Mississauga (CA)

(72) Inventors: Joseph Fisher, Thornhill (CA); Bryan Kowalchuk, Oakville (CA); Bryan Drew Miller, Richmond Hill (CA); Kevin Ramkhelawan, Oakville (CA); Kevin Kowalchuk, Oakville (CA); Cliff Ansel, Thornhill (CA); David Dunn, Toronto (CA); Ludwik Fredorko, Mississauga (CA)

(21) Appl. No.: 12/513,322

(22) PCT Filed: Nov. 7, 2007
(86) PCT No.: PCT/CA07/01998

§ 371 Date: Feb. 21, 2012

Related U.S. Application Data
(60) Provisional application No. 60/863,920, filed on Nov. 1, 2006.

(51) Int. Cl. A61M 16/10; A61M 16/04; A61M 16/22; A61M 16/08; A61M 16/20; A61M 16/00; A61M 16/06

(52) U.S. Cl. CPC A61M 16/1005; A61M 16/0003; A61M 16/0051; A61M 16/0069; A61M 16/0075; A61M 16/0465; A61M 16/0666; A61M 16/0816; A61M 16/101; A61M 16/209; A61M 16/22; A61M 2016/003; A61M 2016/1025; A61M 2202/0208; A61M 2202/0225; A61M 2205/18; A61M 2205/14; A61M 2205/3334; A61M 2205/502
USPC 128/202.22; 128/204.18; 128/204.21; 128/205.13; 128/202.26

(57) ABSTRACT

A portable life support apparatus (1102) and particularly a respiratory support apparatus adapted to be easily mounted to a stretcher is disclosed. Prior devices that attach onto a stretcher are heavy and cumbersome and obstruct access to the patient. Accordingly, there is a great need for a portable emergency support device that overcomes the weight, size, positioning, and other portability disadvantages. One aspect relates to a portable life support device including at least one ambient gas inlet (14); a conditioned gas outlet (30); an oxygen concentrator (26,28) fluidly connected between the at least one gas inlet and the gas outlet, and a ventilator (44) fluidly connected downstream from the oxygen concentrator. A further aspect is directed to a portable life support apparatus in the form of a portable respiratory support apparatus (1102) capable of exploiting both ambient air and expired gas as oxygen sources, wherein the oxygen generator (20) and ventilator (10) are arranged end to end to provide a longitudinal profile that can thus be compactly secured to a stretcher or other similar emergency transport vehicle.

### FIELD OF THE INVENTION

The present invention is directed to a portable life support apparatus and particularly to a respiratory support apparatus adapted to be easily mounted to a stretcher.

### BACKGROUND OF THE INVENTION

When transporting a patient on a stretcher, such as a NATO litter, a large metal bracket called a SMEED is sometimes mounted to the side frame members of the stretcher. The SMEED extends over the patient and serves as a mounting bracket for receiving a plurality of life support devices that function independently of one another. There are several problems associated with the use of the SMEED however. One problem is that the SMEED obstructs access to the patient. Additionally, the SMEED is heavy and cumbersome to use. Loading the SMEED with a variety of different respiratory support and monitoring devices is inefficient from the standpoint of space consumption and weight (the SMEED itself weighs 22 pounds) and does not provide equal optimal access to each of those devices. Accordingly, there is a great need for a portable emergency support device that overcomes the weight, size, positioning, and other portability disadvantages of the SMEED, allows for easy loading of various respiratory support devices in proximity to a subject during the course of emergency transport.

### SUMMARY OF THE INVENTION

In one aspect, the invention is directed to a respiratory support apparatus comprising an oxygen generating device including an ambient air inlet, for generating oxygen from ambient air, at least one gas reservoir, a conduit system for handling gas generated by the oxygen generating device and expired gas exhaled by a patient, wherein the conduit system comprises at least one conduit, operatively associated with a one-way valve, that is fluidly connected between a patient airway interface and the gas reservoir for directing expired gas towards the gas reservoir and at least one conduit, operatively associated with a one-way valve, that is fluidly connected between the gas reservoir and the patient airway interface, for directing reservoir gas towards the patient airway interface. A device according to this aspect of the invention is particularly advantageous for portable applications where size of the oxygen generator and its power consumption are of particular importance.

In one aspect, the invention is directed to a portable combination ventilator and oxygen generator which integrates the functions of producing oxygen and those pertaining to ventilatory support. The inventors have found that controlling oxygen levels supplied to the patient can be accomplished efficiently with superior oxygen generation, conservation and controls and that a patient can be efficiently ventilated in a variety of different types in emergency settings with vastly enhanced oxygen concentrations relative to ambient air in a single portable, weight and size efficient apparatus.

The inventors have determined that a useful arrangement of the gas delivery system is one in which expired gas is collected and re-breathed by the patient, as this potentiates more efficient oxygen output, conserves oxygen already available, and allows the volume and oxygen content of the oxygen generating device to be suited to both ventilate a patient and to be portable i.e. to be of suitable size, weight and power consumption for rapid deployment for a duration suitable in emergency settings. In one respect, since expired gas has a higher concentration of oxygen than ambient air the inventors have found that reuse of oxygen expired by the patient from a previous breath would make generating oxygen feasible within a combination portable unit. Therefore one aspect of the invention provides a portable oxygen generation device that is capable of exploiting both ambient air and expired gas to provide the patients oxygen consumption requirements; the foregoing irrespective of whether the patient is breathing spontaneously or is incapable of breathing spontaneously and is being fully ventilated by an apparatus according to an aspect of the invention.

Accordingly in one aspect the invention is directed to portable respiratory support device comprising a ventilator and an oxygen generating device. In a further embodiment the portable respiratory support device further comprises and an oxygen conservation system adapted to utilize both ambient air and air exhaled by the patient to produce the oxygen requirements of the patient. Optionally, the oxygen conservation system comprises a conduit fluidly connected between the patient airway interface (e.g. a mask) and the ventilator for receiving gas exhaled by the patient and a carbon dioxide scrubber fluidly connected therebetween. Optionally, the oxygen generation device is an oxygen concentrator. The oxygen concentrator may be of the type that is set to operate on a pressure swing adsorption or a pressure/vacuum swing cycle. In another embodiment, the portable respiratory support device further comprises a controller that regulates the oxygen output of the oxygen concentrator based on a sensor system that measures the volume of flow and oxygen gas concentration proximal to the patient inspiratory port.

An embodiment of the invention enables a combined ventilator/oxygen generating device with an output 1.2 L of 90% oxygen or its equivalent. This output has been determined to be adequate to ventilate a patient with 6 litres of 90% oxygen as opposed using an alternative that supplies the entire 6 L of 90% oxygen. For example, for a 2 hour emergency transport mission, the combined weight of the oxygen generator (and scrubber to remove carbon dioxide from the expired gas) suitable for carrying out aspects of the invention herein may be, for example, no greater than 12 pounds (with battery added 15 lbs—with a combined volume of less than 0.27 cu ft, add 1-2 lbs for housing components) and may obviate the need to carry 5 or 6 bottles of oxygen which occupy a much greater volume and weigh far more.

In one embodiment of the invention, the portable life support is adapted to operate in a mode that provides more pleasant ventilation support to a patient capable of breathing spontaneously. Accordingly, optionally, an embodiment of the invention further comprises a by-pass system for by-passing the scrubber. In this connection, the term “by-pass system” or “scrubber-by-pass” is used broadly to refer to any system in which the scrubber is not interposed or in fluid communication between the patient and a gas reservoir on either the inspiratory or expiratory side. It will be appreciated that this can be accomplished with two and three position valves and additional conduits. Optionally, to reduce the size of the apparatus this by-pass system comprises a substitute portion of the breathing circuit, the use of which entails removal of a scrubber unit. Optionally the by-pass system comprises a removable cartridge containing the scrubber and a substitute cartridge comprising a re-breathing circuit, optionally a sequential gas delivery circuit (SGD) which fresh gas is breathed in first and expired gas is available to supply the remainder of the patient's minute ventilation (separate masks can be purchased (Hi-Ox SR) to operate such an SGD with a scrubber unit, where desired). Optionally, the portable life support apparatus is capable of producing fresh gas flow containing approximately an equivalent of 40% patient oxygen sufficient to make up the effective alveolar ventilation of the patient, for example 5 to 8 litres of fresh gas containing 40% oxygen. Accordingly, in one embodiment the oxygen generator is capable producing approximately 2.0 to 2.2 L of 90% oxygen (for example, 2.2 L of 90% oxygen combined with 5.8 litres of ambient air yields 8 litres of 40% oxygen. In another embodiment, the portable life support apparatus is adapted to operate at reduced pressure, for example the atmospheric pressure corresponding to the altitude at which emergency transport helicopters fly for military emergency patient transport. Optionally, to make up 1.2 L an approximate oxygen generator output of equivalent to 1.8 liters of 90% oxygen is required, and an oxygen generator output of equivalent to 3.0 L of 90% oxygen is required in scrubber by-pass mode to effectively make up 2.2 litres of 90% oxygen.

In a further aspect the invention is directed a portable life support apparatus in the form of a portable respiratory support apparatus comprising a ventilator, an oxygen generator and an oxygen conservation system that is capable of exploiting both ambient air and expired gas as oxygen sources with higher oxygen content than ambient air, wherein the oxygen generator and ventilator are arranged (substantially end to end) to provide a longitudinal profile that can thus be compactly secured to a stretcher or other similar emergency transport vehicle. Optionally, the oxygen conservation system is positioned in end to end arrangement with other components. Optionally, the ventilator is positioned between the oxygen generating component and the oxygen conservation system component. Optionally, the oxygen conservation system component comprises at least one of two or more alternative modules (at least a scrubber module which is optionally configured to direct expired gas to the ventilator and optionally a scrubber by-pass which is optionally configured to direct expired gas to an expired gas holding chamber, optionally in the form of an elongated tube and ultimately to atmosphere), for example, in the form of cartridges that have a matching profile to that of the housing the remainder of the apparatus. The apparatus optionally includes a portable power source in the form of rechargeable battery housing unit. Optionally, the portable power source is adapted to minimize the total length of the apparatus and has the same profile as the remainder of the apparatus to make efficient use of profile of the apparatus. Optionally, the placement of the portable power source is at the end of unit opposite end the oxygen conservation system ie. adjacent the oxygen generation device. Optionally, the apparatus further comprises a system for suctioning a patient's airway. In one embodiment the oxygen generator uses a vacuum pump as part of a pressure swing adsorption system to concentrate oxygen from ambient air and negative pressure generated by this pump can be switched (for example with a two or three position valve) between fluid connection to one or more concentrator sieve beds and a suction port, thereby trimming the weight of the combined apparatus relative to separate devices an additional ten to twelve pounds (the weight of a typical standalone suctioning device typically mounted onto a SMEED for patients that may be in need of this form of respiratory support).

In another aspect of the invention, the portable life support apparatus includes a patient monitoring system. The patient monitoring system may display one or more respiratory parameters and optionally displays one or more non-respiratory parameters optionally including ECG, heart rate, continuous or intermittent non-invasive blood pressure, and temperature. The respiratory parameters may be selected from O₂ saturation, expired CO₂ concentration, system CO₂ concentration (particularly immediately upstream of the scrubber) inspired O₂ concentration, airway pressure (particularly, to measure pressure proximal to the patient inspiratory port), respiratory rate, and tidal volume. Optionally, the display also displays one or more device parameters including available battery power and operation mode.

Optionally, the patient monitoring system includes a display that is rotatable between a plurality of viewing positions about a display axis that is parallel to the longitudinal axis of the portable life support apparatus. Optionally, display is shaped to a have a compact longitudinal orientation that complements that of the body of the portable life support apparatus. Optionally, the display axis is positioned between the top and bottom of the display to maximize the rotational range of usable reading orientations of the display. Accordingly, in another aspect, the invention is directed to a portable life support apparatus that integrates in a longitudinally arranged profile within a single apparatus a plurality of respiratory support devices selected from the group comprising an oxygen generator, a ventilator, an oxygen conservation system, an airway suctioning system, and further comprises a patient monitoring system including a display rotatable between a plurality of positions (reading orientation positions) about an axis that is parallel to the longitudinal axis of the portable life support apparatus.

In another aspect, the portable life support apparatus includes a positioning system comprising a clamp assembly adapted to support the apparatus in a horizontal plane parallel to a patient support surface of a portable patient transport apparatus, for example a stretcher. Optionally, the apparatus is supportable proximal to and below the plane of the patient support surface to facilitate access to the patient (first or patient treatment position), as well as above the plane of the patient support surface of the patient transport vehicle (second or patient transport position). Optionally, the positioning system makes the apparatus displaceable between first and second support positions without detachment from the portable patient transport apparatus. Optionally in the second or in a third position the apparatus is partially displaced towards the center of the patient support surface (i.e. at least partially overlying the side support bar of the stretcher or other such vehicle, implicitly to a degree that does not significantly encroach on the side of patient's body). Optionally, depending on the support and loading system accorded to the portable patient transport apparatus within the carrier transport (helicopter, ambulance, humvee or other automotive vehicle), the transport position may also be below the stretcher displaced partially underneath the stretcher.

Accordingly, in another aspect, the invention is directed to a portable life support system comprising a positioning system and a plurality of respiratory support devices integrated in a longitudinal profile into a single apparatus including a patient monitoring system and at least one device selected from the group comprising an oxygen generator, a ventilator and a patient airway suctioning system, the patient monitoring system including a display rotatable between a plurality of viewing positions about an axis that is parallel to the longitudinal axis of the apparatus, the positioning system including a clamp assembly adapted to support the apparatus in a horizontal plane parallel to a patient support surface of a portable patient transport apparatus.

### BRIEF DESCRIPTION OF THE DRAWINGS

The present invention will now be described by way of example only with reference to the attached drawings, in which:

FIG. 1 is a schematic illustration of a life support system in accordance with an embodiment of the present invention, in a ventilation mode;

FIG. 1a is an exploded perspective view of some of the components shown in FIG. 1;

FIG. 2 is a schematic illustration of the life support system illustration in FIG. 1, in a spontaneous breathing mode;

FIG. 2a is an exploded perspective view of some of the components shown in FIG. 2;

FIG. 5 is a perspective view of a portion of a concentrator that is shown in FIG. 15a;

FIG. 6 is a perspective view of a ventilator assembly that is shown in FIG. 15, with a housing element removed and with a bellows shown as transparent for greater clarity;

FIG. 7 is a perspective view of a portion of the ventilator assembly shown in FIG. 6;

FIG. 8 is a perspective view of an end of the life support system shown in FIG. 15, to show in particular a cartridge that is part of the life support system;

FIG. 9 is a perspective view of the cartridge shown in FIG. 8;

FIG. 10 is an exploded perspective view of the end of the life support system shown in FIG. 8;

FIG. 11 is a perspective view of the cartridge shown in FIG. 8 with a transparent exterior to show the inner components and to illustrate gas flow therethrough;

FIG. 12 is a perspective view of the cartridge with a portion of the exterior housing removed;

FIG. 13 is a perspective view from another viewpoint of the cartridge components shown in FIG. 12;

FIG. 14 is a perspective view of the housing component removed from FIGS. 12 and 13;

FIG. 15 is a perspective view of the life support system schematically illustrated in FIG. 1;

FIG. 15a is a perspective view of the life support system shown in FIG. 15, with some housing components removed to show underlying components;

FIG. 16 is a perspective view of a portion of the life support system to illustrate some input that the system optionally receives;

FIG. 17 is a perspective view of a display assembly shown in FIG. 15;

FIG. 18 is a perspective view of a display housing that is part of the display assembly shown in FIG. 17;

FIG. 19 is a sectional view of an end of the display housing shown in FIG. 18, being supported in an end support;

FIG. 19a is a plan view of the end support shown in FIG. 19;

FIG. 20 is a perspective view of the end support shown in FIG. 19;

FIG. 21 is a perspective view of the life support system shown in FIG. 15, with a life support device and an optional positioning system, in accordance with another embodiment of the invention;

FIGS. 22a and 22b are perspective views of a patient transport apparatus connector and a strap, which are part of the positioning system shown in FIG. 21, wherein the patient transport apparatus connector is in an open position;

FIG. 22c is a sectional side view of the patient transport apparatus connector and strap shown in FIGS. 22a and 22b;

FIGS. 23a and 23b are perspective and sectional side views respectively of the patient transport apparatus connector and strap shown in FIGS. 22a and 22b, wherein the patient transport apparatus connector is shown in a patient transport apparatus-connected, strap-unlocked position;

FIGS. 24a and 24b are perspective and sectional side views respectively of the patient transport apparatus connector and strap shown in FIGS. 22a and 22b, wherein the patient transport apparatus connector is shown in a patient transport apparatus-connected, strap-locked position;

FIG. 25a is a side view of a life support device connector shown in FIG. 21, wherein the life support device connector is shown in an unconnected state;

FIG. 25b is a side view of the life support device connector shown in FIG. 25a, with the locking element pushed downwardly;

FIG. 26 is a sectional side view of the patient transport apparatus connector shown in FIGS. 22a and 22b and another life support device connector shown in FIG. 22a, wherein the patient transport apparatus connector is shown connected to a patient transport apparatus and wherein the life support device connector is shown connected to the life support device shown in FIG. 21;

FIG. 27a is an end view of a channel in the housing of the life support device shown in FIG. 21;

FIG. 27b is a plan view of a channel in the housing of the life support device shown in FIG. 21;

FIG. 28a is a perspective view from inside a channel on the housing of the life support device shown in FIG. 21, showing the initial position of a life support device connector during mounting; and

FIG. 28b is a perspective view from inside a channel on the housing of the life support device shown in FIG. 21, showing the final position of a life support device connector after mounting.

### DETAILED DESCRIPTION OF PREFERRED EMBODIMENTS

The following terms are defined as set forth below:

The term “conditioned gas” is used to refer to a gas, optionally conditioned ambient air, having at least one of the following properties: it has a higher content of oxygen than available ambient air, it is less humid than available ambient air, it has a lower nitrogen gas content relative to available ambient air, it comprises exhaled air of a subject that has been scrubbed of carbon dioxide. In a preferred embodiment, the conditioned gas is a gas that has a higher content of oxygen as a result of having been generated by re-breathing circuit and/or an oxygen concentrator and will optionally have been dehumidified and/or scrubbed).

The term “conduit” or “conduit segment” is used broadly to refer to a fluidly intact (pneumatically efficient, and optionally, though not necessarily sealably intact) gas pathway and includes without limitation, tubes and channels of any type that conduct air from place to place.

The term “configure”, “configured” and variations thereof, when used with reference to the capability of an oxygen generating device to generate oxygen, refers to design criteria, that impact on portability including at least one of size, weight, and power consumption of the device in watts/liter of oxygen generated. The term “output controller” used in relation to a controller that controls the oxygen generating device means a controller that controls at least one of: (a) the flow rate of oxygenated gas leaving the oxygen generating device; (b) the concentration of oxygenated gas leaving the oxygen generating device; and (c) the on-off status of the device whereby it can be turned on and off without detrimentally affecting the operation of the apparatus as a whole. This allow the oxygen generating device to be run intermittently to control oxygen concentration and/or power consumption, optionally based on feedback from a sensor that detects the oxygen concentration of gas in the circuit.

The term “conditioned gas outlet” refers to an apparatus outlet or juncture least proximal to the patient that has substantially the final gas composition available for the beginning of the next upcoming patient inspiratory cycle(s). The term “inspiratory gas” is the gas having this composition.

The term “towards” when used describe gas flow in a conduit segment (particularly when in operative association with a one way valve) is used to describe unidirectional flow. It will be appreciated that the location of valves including one way valves and points of attachment of conduit segments may often be dictated by convenience or certain advantages which are not necessarily critical to the operation of the structure in which they are incorporated. Accordingly, precise structural linkages may not be material to the operation even if specified in a drawing or descriptions of preferred embodiments of the invention and equivalent arrangements will apparent to persons skilled in art. The term “operative association” and related terms are meant to signify that the precise method of association or location can be variably selected without inventive skill and do not materially affect the operation of some embodiments of the invention. It will also be appreciated that portions of the gas circuit may be left outside the body of the apparatus, particularly disposable, relatively inexpensive, commonly replacable and technologically trivial parts, and connected by the user via a port designated for such connection, in effect making the port equivalent to those portions of the gas circuit, if added after and secondary to the essential features of the apparatus. Persons skilled in the art of working with respiratory apparatus are attuned to assembly of these types of circuit elements and will readily perceive an assembly of parts as the essential apparatus. Examples follow. A scrubber may be introduced into the circuit outside the core apparatus and may be so introduced advantageously on the inspiratory side of the circuit but without very significant effect also on the expiratory side of the circuit. Oxygenated gas leaving the oxygen concentrator may be introduced into the conduit system or directly into the ventilator reservoir.

The term “ventilator” includes pressure based ventilators that provide pressure to the airway of the subject to a certain preset level (e.g. 25 cm H2O) or range, and volume based ventilators that control the tidal volume and frequency of the inspiratory flow to the patient. Ventilators of these types could be used for ventilatory assistance of a type that does not require rigorous pressure, volume, frequency controls. A variety of types of ventilatory assistance are known to those skilled in the art including CPAP, BiPAP, pressure controlled, volume controlled, pressure support ventilation, airway pressure release ventilation, inspiratory pause, inspiratory flow profile, proportional assist ventilation, neurally activated ventilatory assistance, assist control ventilation etc. The term “ventilator device” is used broadly to refer to a ventilator and may depending on the context implicitly exclude the gas reservoir component of such a device.

The term “oxygenated” means air having an oxygen content higher than ambient air, optionally having a concentration of at least 40% oxygen.

The term “portable life support apparatus” (or interchangeably “portable life support device”) as used herein, generally is used to refer to the apparatus as whole the name contemplating but not implying monitoring functions that are not limited to respiratory parameters. However, this term may be used interchangeably with “portable respiratory support apparatus” and “respiratory support apparatus”, among others, in which the primary functions of respiratory support are highlighted in name.

The term “substantially in series” with reference to longitudinal configuration of an apparatus or system according to an embodiment of the invention is not meant to necessarily imply that each component, of each sub-assembly—namely oxygen generator, ventilator mechanism, scrubber unit, battery housing is in a distinct compartment with each compartment arranged in series but rather that the most space consuming part of each sub-assembly is arranged in series in a longitudinal configuration.

The term “re-breathing circuit” means any circuit in which exhaled air is captured and remains available for re-breathing during a portion of an inspiratory phase of breathing. The re-breathing circuit may be optionally a sequential gas delivery (“SGD”) circuit. The term sequential gas delivery circuit means a system which includes controls, (for example, valves) that are set to open sequentially, for ensuring that in the first part of an inspiratory cycle the patient receives a gas of a first composition and in the second part of the inspiratory cycle, the patient receives a second different composition (for example the first gas may be oxygen and the second gas may be gas exhaled from a previous inspiratory cycle). The term “a sequential gas delivery valve” means a one way valve set to open to source of expired gas which opens at a higher pressure than a valve set to open to a source of fresh gas and which is typically positioned in parallel to an expiratory valve which allow expired gas to leave to ambient air. A “sequential gas delivery control system”, optionally a valve system, refers to at least two valves that open sequentially to fresh gas and expired gas sources, typically valves that open at different pressures, one at a lower pressure connected to an inspiratory gas source, and one at a higher pressure connected to an expired gas source.

The term “fresh gas” generally means gas entering the patient's breathing circuit that does not contain appreciable amounts of carbon dioxide, and is usually air or oxygen enriched air, although other components may be present as well, such as anesthetic agents or the like.

The term “inspiratory relief valve” means a valve that allows gas, usually ambient air, into a portion of the conduit assembly that is available to the patient to breathe on during an inspiratory cycle in which inspiratory gas, usually in the form of a conditioned gas, is temporarily depleted.

The term “patient airway interface” means a patient interface such as a mask, nasal tube, endotracheal tube, or tracheotomy tube that is fluidly connected to a patient airway.

The term “independently movable” is understood to mean having some degree of independent movement, for example, rotational independence about at least one axis.

The term “reading orientation” means the preferred orientation in which a line of data, normally readable in a horizontal orientation (in the case of many languages) from left to right (or right to left), for example a number, is presented horizontally and therefore is most easily readable. By contrast, a “reading position” is any screen position in which substantially all of the data is normally viewable by a user, though not necessarily in a reading orientation.

The top and bottom with reference to the display refer to the borders of the display parallel to its reading orientation.

The term “intermediate” with reference to a rotational position refers to a position roughly in the middle of its rotational extremes.

The term “vertical” with reference to a display position means that the screen axis is roughly parallel to the ground and the display is a plane roughly perpendicular to the ground.

The term “airway” includes, without limitation, the mouth, trachea, and nose.

The term “processing” with reference to machine intelligence means any handling, merging, sorting or computing of machine readable information using digital or analog circuitry in a way that it is compatible with visual presentation on a screen.

The term “reading” is meant to include scanning with a view to interpretation of any visually depicted subject matter (not just letters and numbers), including without limitation, graphs, symbols, heart monitor output, brain waves etc. Optionally the device is considered positionable for reading when the device is anywhere within arms length of the user, so that the user can use the user interface on the display.

The term “plane” is used broadly to include a curvilinear profile that is substantially planar.

The term “preferred position” means a position with reference to the position of the portable life support device about an axis generally parallel to the axis of rotation of the display that permits use of at least half and preferably substantially more of the range of motion of the display.

The term “device positioning interface”, alternately called the “device interface” when referencing the positioning system, is used broadly to refer to any interface that serves as a point of attachment of the support structure of the positioning system including, without limitation, a flat metallic surface for engaging a magnet.

The term “holding” with reference to a way of keeping the screen from rotating is understood in its broadest sense to provide resistance to rotation. For example, the apparatus may include a spring-loaded detent that interacts with a plurality of grooves defined by annularly located toothed portion of the screen housing, so that rotation of the screen in one direction or the other successively engages the intermittent grooves defined by those teeth to define a series of intermittently spaced screen positions.

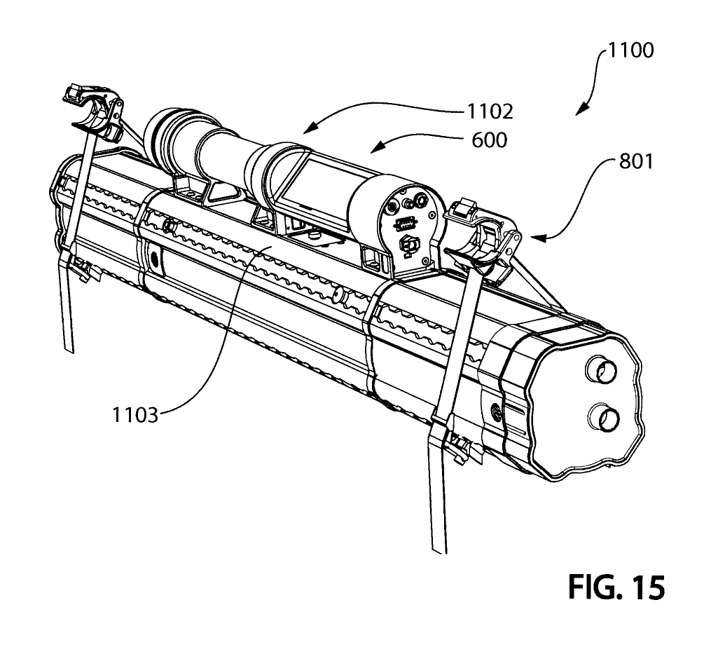

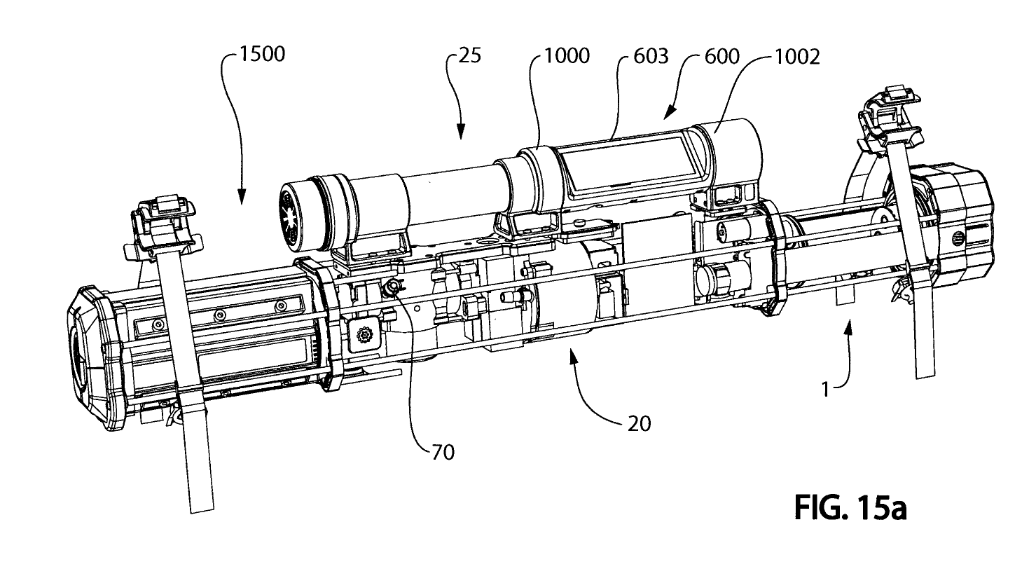

As detailed below, and shown in the drawings (FIGS. 15 and 15a), the portable life support system in accordance with an embodiment of the invention 1100 includes a portable life support apparatus 1102 and positioning system 801 optionally including an independently movable display 600 (also termed a readable output display) that maximizes the versatility of the positioning system by enabling the life support device to be positioned in a variety of convenient positions in which the display can be independently positioned to be readable. This positioning system is useful for positioning a variety of emergency treatment/monitoring devices in relation to portable patient ambulatory vehicles such as stretchers and rolling beds used in civilian applications.

As detailed below, according to one embodiment of the invention, the portable life support system is a portable respiratory support apparatus that provides treatment in the form of ventilation and oxygen supply and may be used in many emergency and medical transport applications, particularly by the military—for example, in the field or in transit between forward field surgical units and more permanent treatment units (Echelon 3 units); as well as in the civilian market for emergency transport by ambulance and helicopter. This unit optionally includes a facility to suction the airway of a subject.

In one aspect, the portable life support system serves to monitor the outcome of respiratory treatment parameters and also serves to monitor non-treatment parameters of importance to attending medical personnel such as the patient's ECG, heart rate, temperature and blood pressure. Device parameters may also be displayed most notably available battery power and operation modes. Respiratory treatment parameters measured and displayed by the life support system are detailed below. In a general aspect, the portable life support system of the invention contemplates that other forms of treatment and/or monitoring could be provided, measured and/or displayed.

The term “treatment” is used broadly to refer to ministrations of any kind, including without limitation provision of respiratory gases, drugs, stimuli, signals etc.

A preferred embodiment of the invention will now be described, and relates to a portable respiratory support apparatus and measurement of respiratory physiological parameters for display.

The portable respiratory system optionally comprises six main components collectively termed Mobile Oxygen, Ventilation and External Suction system (portable life support apparatus 1102) Referring to FIG. 15a:

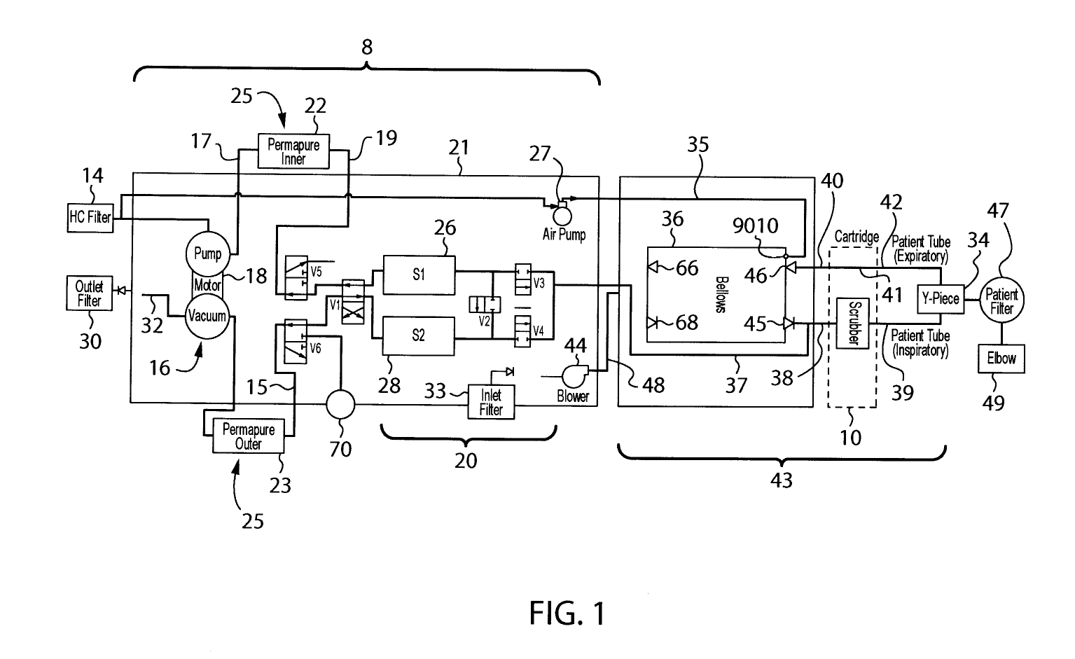

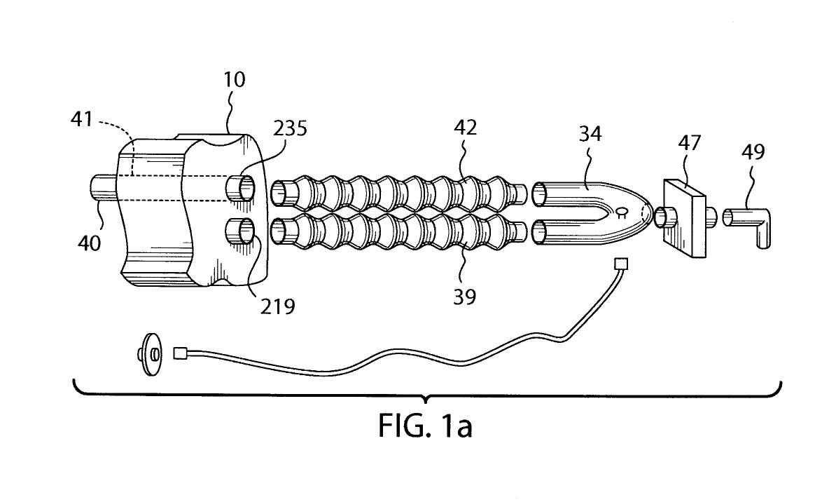

A “conditioning section” 8 (FIG. 1) for ambient air, including a source of O2, in one preferred embodiment an oxygen concentrator 20, optionally including a dehumidification device 25 (optionally in the form of a moisture exchanger);

a ventilator 1;

a patient airway suction system including a suction port 70, which is used with conventional accessories to clear the patient's airway;

a patient monitoring system, in the form of a display 600, which monitors and displays a patient's vital statistics and optionally device parameters, for example remaining battery power, tidal volume (in ventilatory mode);

the breathing circuit, which may optionally be a circle circuit, a non-rebreathing circuit, or a partial rebreathing circuit, which controls delivery of gas to the patient and which may optionally operate in either a ventilated or spontaneously breathing mode, and

a power system 1500, which may for example optionally be comprised of rechargeable batteries, or removable, rechargeable batteries, or removable hot swappable batteries, and which may optionally include an AC power supply, or which may optionally including a connector for connecting to an AC power supply.

In one aspect of the invention the oxygen content of the system is controlled independently of the minute ventilation of the patient, that is, without reference to the patient's minute ventilation and the operation of the ventilator. Accordingly, the inventors have discerned that integrating emergency ventilation and oxygen supply functions is simplified and energy efficient according to the invention. In one aspect of the invention, the oxygen supply is controlled in response to the patient's minute oxygen consumption. This may be done by measurement of oxygen concentration in the system, for example with an oxygen sensor, and turning the oxygen supply on only when the oxygen concentration drops below a set value, and turning the supply off when it reaches a set concentration.

For example, when the oxygen concentration reaches 85%, a controller operatively connected to sensor can shut the concentrator off. When the oxygen concentration is sensed to fall to 80%, for example, the oxygen concentrator can be turned on again. This minimizes the amount of oxygen that needs to be produced by the system and hence provides for a more energy efficient and lightweight system.

Importantly, at least some embodiments of the invention overcome dismissive perspectives attributable to presumed design constraints on using an oxygen concentrator in place of oxygen tanks, in emergency transport settings, in terms of power consumption, required size and output of O₂. Importantly, at least some embodiments of the invention contemplate that these constraints can efficiently be obviated in important part by re-circulating a patient's expired gas in a circle circuit, or partially reusing a patient's exhaled gas in a partial rebreathing circuit, for example a SGD circuit. In particular, since ambient air traditionally conditioned in concentrators contains 21% O₂ and exhaled gas of patients receiving, for example, 40% O2, contains approximately 33-35% O2, it has been determined according to an embodiment of the invention to be efficient as well as otherwise advantageous, to use an O₂ concentrator in conjunction with a circle or re-breathing circuit and scrubber, to supply oxygen to patients requiring ventilation during emergency transport. Some embodiments of the invention also contemplates that patients not requiring ventilation can also be efficiently supplied with oxygen generated by an oxygen concentrator by providing a reservoir for collecting the patient's exhaled gas and optionally allowing the patient to use exhaled gas at the end of an inspiratory cycle.

As generally shown in FIGS. 1, 2 and 15a, according to a preferred embodiment of the invention, the concentrator 20, ventilator 1 and re-breathing sections of the portable life support system are generally arranged end to end, to generate a compact longitudinal profile that matches that of the stretcher (litter) or other similar portable vehicles that support patients in a reclining position. This profile is particularly advantageous in at least the following respects:

Litter support stanchions in military helicopters have at least three levels at which litter support hooks or shelf like supports are located so that at least three litters may be occupied and supported one on top of the other during emergency transport from a small surgical field unit (for example a unit that may have only one surgical table, one pre-op area and one post-surgical monitoring area nearest the combat zone—sometimes known as a Forward Resuscitative Surgical Site (FRSS) or Echelon 2 facility) to the next more permanent or Echelon 3 medical facility. The longitudinal profile best enables the portable life support apparatus to be suspended parallel to the litter, off its side, or above it with minimal interference to access to the patient to whom the unit is allocated or any patient above or beneath.

The portable life support apparatus can be supported in at least two positions longitudinally displaced from another with individual positioning structures that do not need to support the entire weight of the apparatus and are hence simpler, more versatile, lighter and less bulky.

The screen displaying vital statistics may be compactly oriented in a longitudinal orientation parallel of the orientation of the life support apparatus. In this orientation, the display may be adapted to rotate into a variety of planes about an axis parallel to the axis of the apparatus so that it can be rotated into a reading position that accommodates upper, intermediate and lower litter positions.

According to one embodiment of the invention, the portable life support apparatus treats both spontaneously breathing patients and ventilated patients and may be operated differently in “spontaneous” mode versus “ventilation” mode, as described below.

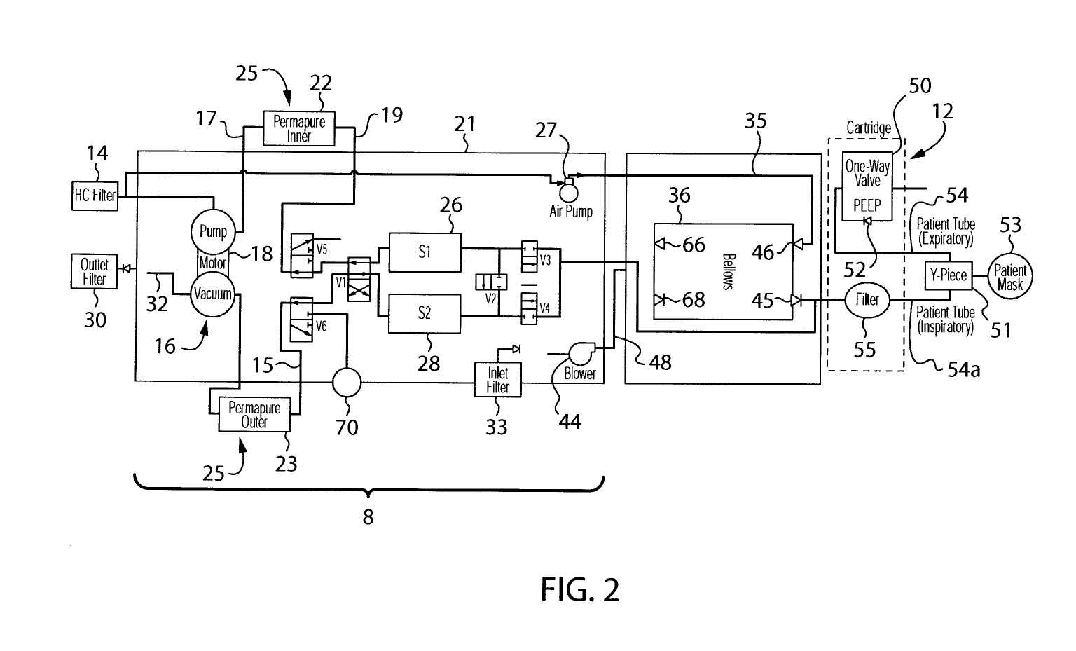

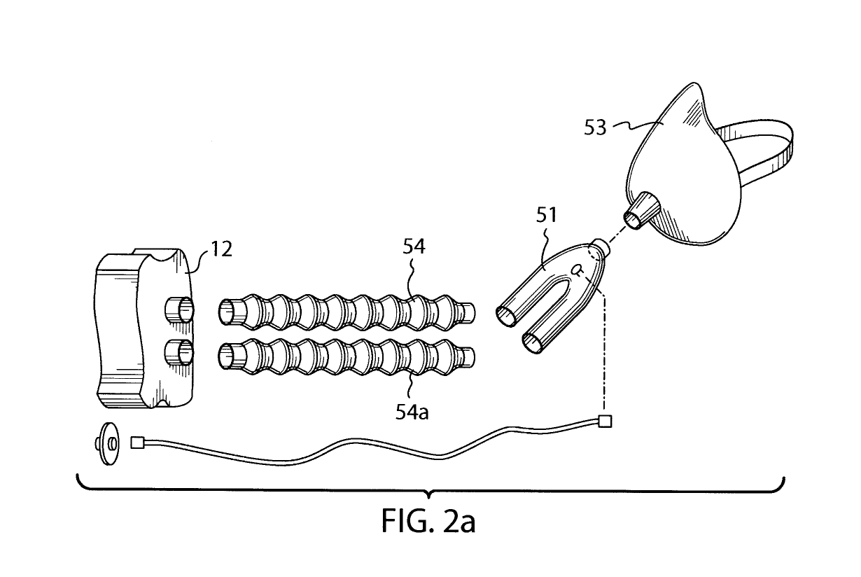

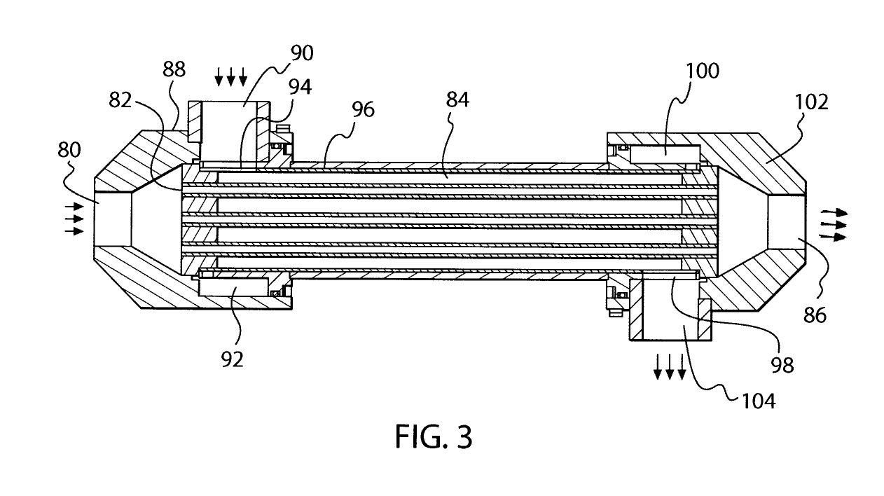

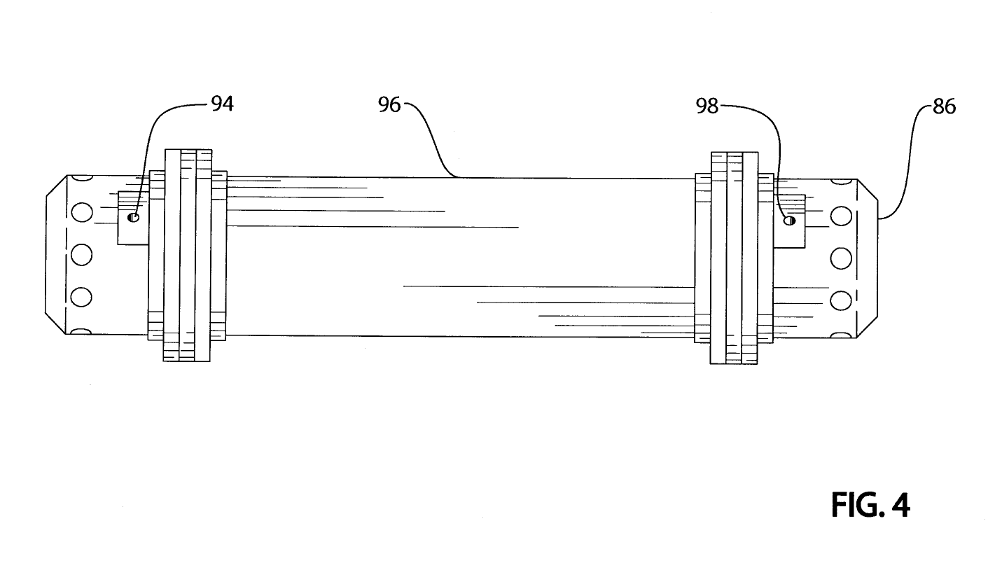

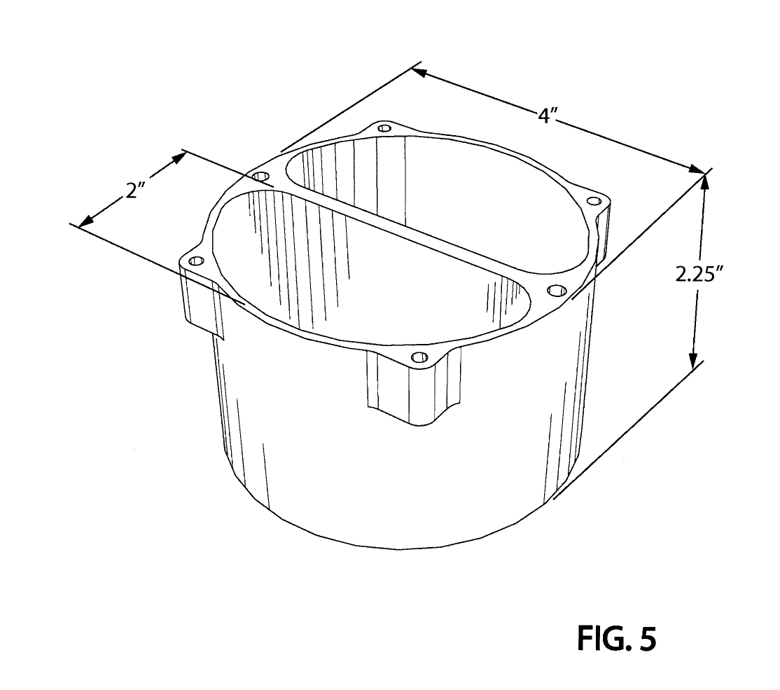

Referring to FIGS. 2 and 2a, with respect to the spontaneous mode of operation, it is first particularly noteworthy that tubes 54, 54a serve to distance the expiratory outlet 50 contained in cartridge 12 from the patient mask (or other equivalent patient airway interface), which interface does not have an expiratory port, so that oxygen enriched gas is not lost from the mask.

Furthermore, as shown in FIG. 2, inasmuch as cartridge 12 may include a sequential re-breathing valve 52, the afore-exemplified mask may be attached to the fresh (also termed “conditioned”) gas output by a length of conduit that stores exhaled gas—for re-breathing in conjunction with a planned requirement for inspiratory relief, prior to the end of an inspiratory cycle, making the oxygen production requirements of the concentrator even more adaptable for portable emergency use.

As shown in FIGS. 1 and 2, schematically describing embodiments of the “ventilated” and “spontaneously breathing” or “spontaneous” modes, respectively, the system may be modularly constructed to provide a separate ventilator cartridge 10 for attachment to the device for operation in a “ventilator” mode and a different cartridge 12 for operation in the “spontaneous” mode.

In both ventilated and spontaneous modes, ambient air enters the portable life support system, through the hydrocarbon filter 14 drawn by the pump 16 and through “conditioning” section generally identified as 8 (oxygenation and optionally dehumidification) of the conduit assembly, as described hereafter. The pump/vacuum 16 (optionally combined) pumps ambient air via conduit section 17 through the inner tubes 82 (shown in FIG. 3) of a dehumidifier, optionally a moisture exchanger 25, where it encounters lower pressure lower humidity gas being purged from the concentrator 20. Dehumidified air leaves the moisture exchanger 25 through conduit section 19. Valve V5 determines when the dehumidified air is used to pressurize one of the sieves 26 or 28. During part of the concentrating cycle, for example when valve V2 is open for one sieve to prime the other (as described below), V5 directs the air into the concentrator housing to cool the motor 18 and sieves 26 and 28 and other components within first longitudinal section of the device e.g various control boards not shown and air pump 27). Valve V1 determines which sieve is being pressurized and which is being vacuumed. The vacuum head of the pump 16 draws the dry nitrogen-enriched air from the sieve S1 or S2 being purged through the valve V1 and directs it via conduit section 15 through the outer counter flow part 23 of the moisture exchanger 25 around the tubes 82 (see FIG. 3) where it is used to dehumidify the inbound air from conduit section 17. The vacuumed air exits the device via conduit section 32 and outlet filter 30. Air intake through hydrocarbon filter 14 is also pumped through conduit section 21 via air pump 27 and then into the inspiratory reservoir 36, optionally a bellows, via bellows entry port 9010. Air received via air pump 27 is primarily needed for mixing ambient air with oxygenated air for delivery to spontaneously breathing patients. For example, spontaneously breathing patients may receive eight litres per minute of fresh gas flow composed of a combination of 5.8 litres of ambient air and 2.2 litres of 90% oxygenated air (combined in the conduit 38) so that they get fresh gas with 40% oxygen. Although a supplementary volume of air is not needed in the ventilated mode of operation, in some instances, it may be expedient to mix some ambient air into an over-oxygenated air stream.

It is generally understood that there are a variety of ways of attenuating the oxygen concentration, in either ventilated or spontaneous mode, depending on the fresh gas flow and oxygen concentration requirements of the patient. These include shutting the concentrator off for a period, blending the oxygenated air with ambient air or changing the oxygen concentration settings (for example, with a controller that controls parameters affecting the performance of the concentrator such as working pressures and length of the cycles).

In a circle circuit, the O₂ concentration and rate of the fresh gas flow (“FGF”) are set so as to provide at least the oxygen consumption of the patient, which may be only 200-300 ml of O₂ per minute, at a concentration determined by the needs of the patient. If the FGF has a concentration of 85%, then only 350 ml/minute of FGF is required. However in practice it is common to provide a higher rate FGF, for example, at least 1 L/min to flush out trace gases from the system. By way of example, assume the concentrator is capable of providing 2 L/min of 85% O2, and only 40% O2 is required for a particular patient with an oxygen consumption of 300 ml/min. The minimum FGF for this patient would be 500 ml/min of 40%. If 1 L/min of FGF is required to flush out the system, the 40% concentration may be generated by running the concentrator produce 85% intermittently (for example, the concentrator would be run with a 15% duty cycle to produce 0.3 LPM of 85%) and blending with 0.7 LPM ambient air to achieve 1 LPM at 40% oxygen concentration. Alternatively, the concentrator working pressures may be adjusted to produce 1 L/min of 40% oxygen without blending with ambient air.

As shown in FIG. 1, the conduit assembly of the portable life support apparatus 1102 may comprise a semi-closed “circle circuit” generally identified by 43 comprising conduit inspiratory sections 38 and 39 and expiratory sections 40, 41 and 42, all fluidly connected to an inspiratory reservoir 36. As shown in FIG. 2, the conduit assembly of the portable life support apparatus 1102 may comprise a partial rebreathing SGD circuit comprising conduit inspiratory sections 38 and 54a and expiratory sections fluidly connected to an inspiratory reservoir 36, and expiratory sections 54, 50 leading to ambient air.

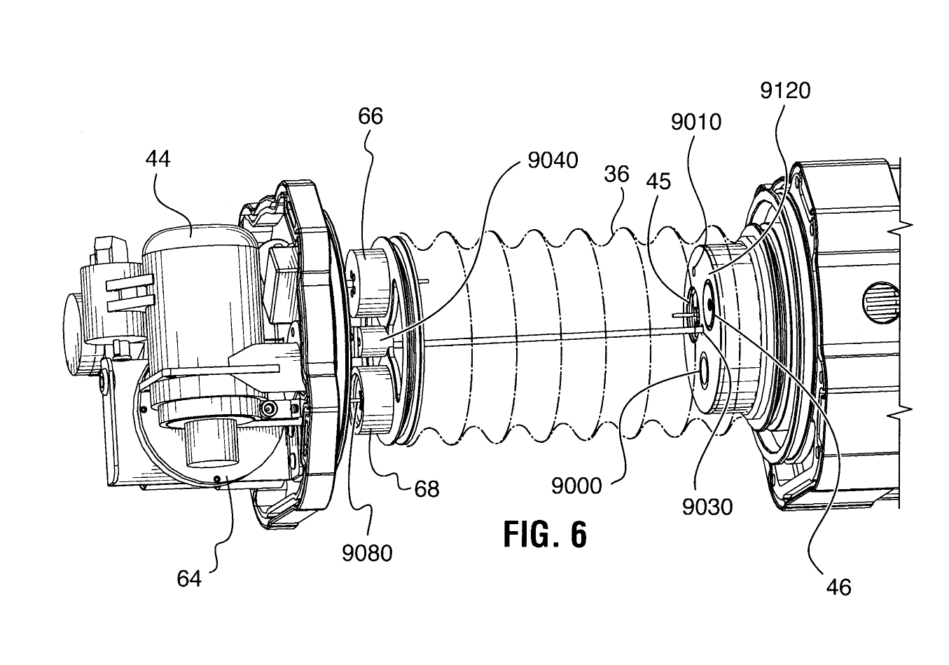

In one embodiment of the invention, the inspiratory reservoir 36 takes the form of a bellows that is acted on by blower 44 (receiving air through conduit section 48) to exert positive inspiratory pressure to ventilate the patient. The bellows 36 contains an expiratory valve 66, for example a positional valve (see FIG. 6) that only opens when the bellows is fully expanded; when it is fully expanded it may only require a pressure of 1 to 2 cm of H₂O to maintain the bellows in a position where the valve is positionally open. On the inspiratory side the inspiratory relief valve 68 may also be a positional valve that opens when the bellows is fully contracted and is set to open, at a pressure which ensures that the blower is operating efficiently (working the bellows and not the valve). In the spontaneous mode, it is desirable to have rebreathing valve 52 open when bellows 36 is depleted and the subject is still inspiring, in preference to having inspiratory relief valve 68 open. Thus, the opening pressure of inspiratory relief valve 68 is preferably set higher, and preferably at least 1-3 cm H₂O higher, than rebreathing valve 52. A one-way valve 45 (set to open easily—e.g. 0.5 cm of H₂O) leads from the bellows to the patient, via either the CO₂ scrubber cartridge 10 (shown in FIGS. 1, 9 to 11, in particular), or the spontaneous breathing cartridge 12. Conduit section 38 receives a combination of oxygenated air from the concentrator via conduit section 37 and from the bellows 36. Bellows 36 which is normally filled: a) with exhaled air (in the ventilatory mode) carried to the bellows via conduit sections 40, 41 (a scrubber by-pass path directly through the cartridge 12 as shown in FIGS. 1, 9-11) and 42; and b) with ambient air received from air pump 27 via conduit section 35, primarily in the spontaneous breathing mode (in the spontaneous breathing mode exhaled air enters the atmosphere through a one way valve 50 (set to open easily—e.g. 0.5 cm of H₂O).

In an alternative embodiment, the inspiratory reservoir 36 could comprise some other suitable vessel, such as a bag, instead of a bellows.

Conduit 38 leads to the patient via inspiratory hose 39, optionally an extendable hose, through a Y-piece 34 that connects (in ventilatory mode) to a patient's endotracheal tube (not shown) through a filter 47 via an elbow connector 49.

On the expiratory side, in ventilatory mode, Y-piece 34 is connected to expiratory conduit sections 42, 41 and 40, and through one-way valve 46 (set to open easily—e.g. 0.5 cm of H₂O) to the bellows 36.

By contrast, in spontaneous breathing mode, cartridge 12 receives expired air through patient expiratory conduit section 54 which leads to a one-way valve 50 (set to open easily—e.g. 0.5 cm of H₂O) to atmosphere and sequential rebreathing valve 52 (e.g. set to open at 2.5 cm of H₂O) which may be planned to open during a planned re-breathing part of the inspiratory cycle and is generally triggered to open during the latter portion of inspiration when the patient's breathing rate exceeds the rate of fresh gas flow.

As described above, expiratory reservoir in the form of optionally extendable expiratory hose 54 one way valve 50 provides a point of venting expired air to atmosphere at a distance from the patient mask, so that much of the 8 litres of 40% oxygen typically generated for a spontaneously breathing patient in need of oxygen, is not otherwise immediately lost to atmosphere via an expiratory vent in the mask. Expiratory hose 54 optionally contains at least 200 ml of volume.

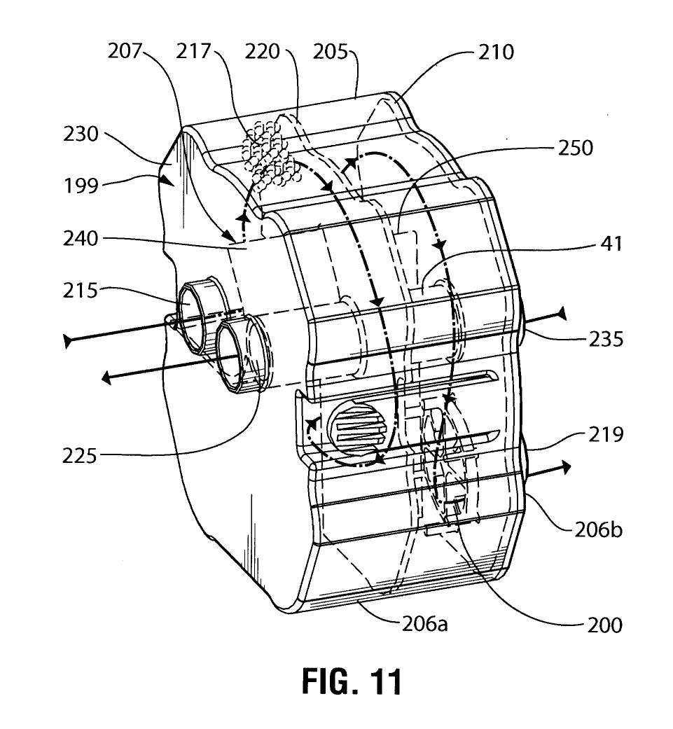

The ventilator cartridge contains a CO₂ scrubbing material (e.g. soda lime). Inasmuch as portability may often entail size limitations and hence possibly longitudinal space limitations that reduce the path length through which expired gas can travel for scrubbing (reducing the amount of CO2 that can be removed by the scrubber), a scrubber design containing a helical scrubber material chamber/airflow pathway, as particularly shown in FIGS. 11 to 13, can be used to increase the path length and amount of CO2 that can be removed from the patient's expiratory gas.

As described above, ambient air enters the circuit through a hydrocarbon filter 14 and is optionally pumped by pump 16 (having a common motor 18 with vacuum 20) directly into the core of moisture removal device 22 which is generally configured like a shell and tube heat exchanger, as shown in FIG. 3.

In one embodiment, as shown in FIG. 3, ambient air is directed through inlet 80 and outlet 86 (Path A) which are fluidly connected to tubes 82, optionally made of Nafion®, a material known for its moisture (water molecule) permeability properties, and utility in removal of moisture from a current of air, using a counter flow gas of lower humidity. As shown in FIG. 3, ambient air flowing through the tubes 82 may be conditioned in the core 84 of the de-humidification device with lower humidity, lower pressure, air traveling around the tubes via path B (inlet 90 to outlet 104). The dehumidified gas exits through core outlet 86. The counter flow or “conditioning air” enters the core through the inlet-sleeve 88 via inlet-aperture 90 in the core wall 96 (also shown in FIG. 4) and travels through a circumferential sleeve-defined pathway 92 around the outside of the core. Pathway 92 is in fluid communication with the counter flow pathways through and around the outside of tubes in the core 84 via air-dispersing, screen-like openings 94 around the periphery of the core wall 96. Counter flow air exit through openings 98 which are in fluid communication with exit-sleeve pathway 100 defined by exit-sleeve 102 that lead to exit aperture 104.

As shown in FIGS. 1 and 2, in one embodiment of the invention, air vacuumed from the sieve beds 26 (S1) and 28 (S2) of the oxygen concentrator 20, is vacuumed alternatively from sieve bed 26 and 28 through valve V1, which is in alternate fluid communication with S1 and S2 and exits into atmosphere through valve V6 and vacuum 16 through outlet conduit 32 past outlet filter 30. The vacuum 20 purges the sieve beds 26 and 28 in alternating cycles described immediately below. This purged air is enriched in nitrogen gas adsorbed by the zeolite material (e.g. Oxysiv®) in the sieve beds and is at substantially lower pressure than the ambient air pumped through Path A. This purged air is used as the low-pressure, lower humidity, counter flow gas to remove humidity from ambient air.

De-humidifiers of the type sold under the name Perma Pure® e.g. FC series handling flow rates of up to 80 slpm, for example 75 slpm, can be used in the present context for gas-gas dehumidification. Optional adaptations of off-the-shelf Perma Pure specifications (e.g. FC-125), include increased Nafion® membrane thickness (e.g. to 0.030 in.) to handle larger pressure differentials between ambient and counter flow gases, and increasing tube number (e.g. to 400 tubes). Parameters impacting on dehumidification include the differences in humidity and pressure between the gas inside and outside the tubes. The combined effect of changes in humidity and pressure differences across the tubes can be routinely approximated using published data and can be readily empirically determined. In one embodiment of the invention, the pressure differential in the tubes is approximately 36 psi.

According to one embodiment of the invention, the oxygen concentrator is of the type that operates on a pressure swing adsorption or a pressure/vacuum swing cycle as described by way of background in U.S. Pat. No. 6,478,850 (the '850 patent), the contents of which are hereby incorporated by reference. As described in the '850 patent, the concentrated oxygen is released to the breathing circuit following which some is used to prime the second sieve bed. The sieve bed is then vacuumed to release the nitrogen and purged. This occurs in repeating cycles, with each sieve bed alternately being pressurized, releasing oxygen to the breathing circuit, then being purged. As shown in FIGS. 1 and 2, the two sieve beds 26 and 28 (S1 and S2) may be alternatively pressurized and purged with a valve V2 between the two sieves permitting recoup of a partially concentrated gas for use in the subsequent pressurization cycle (in the other sieve). Oxygen enriched gas is released from S1 via valve V3 and from S2 via valve V4. In one embodiment of the invention, the O₂ concentrator produces at least 2.2 L (at standard pressure) of 90% O₂.

According to one embodiment of the invention, the portable life support system comprises a volume controlled ventilator that provides control of respiratory rate, tidal (breath) volume, and delivered oxygen concentration. As shown in more detail in FIG. 6, the ventilator is comprised of a blower 44, a sealed container 35 that houses the inspiratory reservoir in the form of bellows 36 and a volume measurement device, for example a positional encoder that measures displacement of the bellows via string 9061.

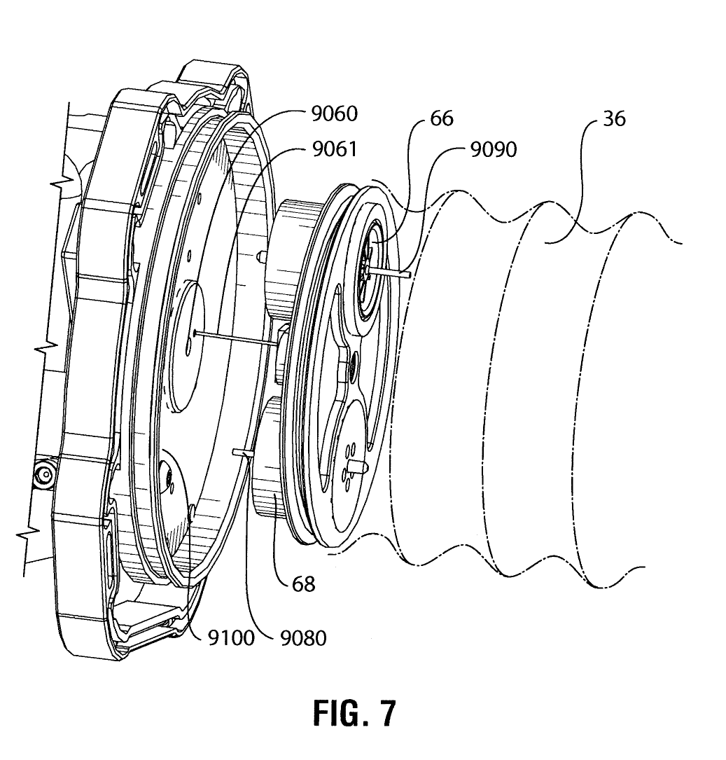

As shown in FIGS. 6 and 7, representing a detailed view of the ventilator and positional inspiratory and expiratory relief valves 66 and 68, respectively, one way inspiratory valve 45 and one way expiratory valve 46 communicate with the interior of the bellows 36. An oxygen sensor port 9000 (communicating with an oxygen sensor—not shown e.g. MiniOx®) may be used to sample the oxygen concentration in the bellows. The bellows 36 is also in fluid communication with conduit section 35 carrying ambient air propelled by air pump 27, via bellows entry port 9010. The bellows may be set to move along a rod 905, which is positioned via aperture 9030 and 9060 and bellows sleeve 9040. Pin 9080 on positional expiratory relief valve 68 makes contact to open when the bellows is fully expanded, enabling pin 9080 to contact bulkhead surface 9100, whereupon sufficient expiratory pressure to maintain pin contact with the bulkhead surface will provide expiratory relief. Similarly, pin 9090 of positional inspiratory relief valve 66 will contact projecting surface 9120 of the bellows floor to open inspiratory relief valve 66 in a ventilated mode of operation which is set to open at a pressure e.g. 5 cm H20 (greater than the inspiratory relief valve—within cartridge 12—optionally set at 2.5 cm H20).

To vary the delivered oxygen concentration ambient air is pulled through the hydrocarbon filter by the air mixing pump and delivered to the patient expiratory tube 40 or to the bellows 36. This air fills the bellows and is mixed with concentrated product to deliver desired oxygen concentrations (for example, 40% and 85%).

During ventilation, the blower produces the pressure that forces the bellows to collapse delivering whatever gas blend is in the circuit to the patient through the scrubber, at the desired tidal volumes and respiratory rates. Correct estimation of the tidal volume requires adjustment for the concentrator generated enriched oxygen flow, that is not simply measured by displacement of the bellows. The blower 44 draws air through the inlet filter 33 and delivers it to the sealed chamber housing the bellows 36.

As shown in FIG. 1, during patient exhalation, expired gas travels down the expiratory tube 42, 41 and 40 through a one-way valve 46 into the bellows. When the volume in the bellows exceeds maximum volume, excess gas is expelled via the expiratory vent 66 into the bellows chamber, which exits the chamber through the blower. Since expired gas exits through the blower, running the blower during exhalation at a controlled constant rate provides positive end expiratory pressure (PEEP). The PEEP may be controlled, for example to provide varying levels between 0 and 20 cm H₂O. Additionally, in the breathing circuit, a safety pressure relief valve (not shown) is located with an opening pressure approximately equal to the maximum desired airway pressure, for example, 60 cm H₂O. Optional ranges for ventilation parameters include:

1. inspired O₂ concentrations of 21% to 93%—For increased ease of use, 3 presets may be settable by the user of 21%, 40%, and 85%. Tidal volumes may be settable between 400 ml and 1 litre (e.g in increments of 100 ml), which are useful for adult ventilation.

2. Breath Frequency: between 8 and 20 per minute

3. PEEP: 0-25 cm H₂O optionally with settings incremented in 5 cm H₂O

4. Inspiratory: Expiratory ratio between 1:1 AND 1:2—this is typically adjusted automatically based on tidal volume, breath frequency, and blower flow rate.

5. End Inspiratory or end expiratory Pause with pressure hold.

If the system reaches the maximum airway pressure limit set on the ventilator control, the blower stops blowing and switches into constant PEEP mode as described below.

The system is able provide intermittent tracheal suction. A suction kit consisting of a wand, hoses, and suction bucket with optional filter may be attached to the suction port. Activating suction mode energizes valve V6 which then connects the vacuum head of the pump 16 to the suction port 70. The vacuumed air is then vented through the outlet filter 30. Suction is preferably at approximately 100 mm Hg but may be higher or lower is dictated by the patient's requirements. A suction relief valve (not shown) may be optionally provided in parallel to the suction path and leads to ambient air to ensure the suction does not exceed the maximum desired vacuum level.

As shown in detail in FIG. 6, the spontaneous breathing cartridge 12 may be attached for the device to operate in spontaneous breathing mode. The cartridge contains an optional patient filter 55 (which can prevent patient secretions from entering the bellows) and may contain two valves 50,52 to allow sequential gas delivery, for example as described in WO/2004/073779. Expiratory valve 50 leads to ambient air through port 7420. When the inspiratory volume of the bellows is depleted, if the patient is still inspiring, valve 52 opens permitting rebreathing of expired gas contained within a length of patient tube 54 (for example six feet). Optionally, an additional exhaled gas reservoir, such as a rebreathing bag, may be connected to port 7420.

In spontaneous breathing mode the concentrator works exactly the same as in ventilated mode. The oxygen produced by the concentrator is fed to the inspiratory limb 37 of the breathing circuit. In ventilated mode, the ventilator and concentrator controls preferably communicate so that oxygen is not released from the concentrator to the breathing circuit during the last portion of inspiration, as this volume would not be accounted for in the tidal volume measurement, as determined for example by the bellows position sensor.

As described above with reference to FIG. 2, ambient air is delivered to the bellows 36 from the air pump 27. The air pump draws its air from ambient via the hydrocarbon filter 14. The rate of ambient air entrainment is approximately 5.8 LPM, which when mixed with 2.2 LPM of 90% O₂ provides 8 LPM of 40% O₂, sufficient to meet the alveolar ventilation requirements of most patients at rest. The concentration of oxygen that is achievable by the system depends on the capacity of the concentrator and on the ventilation requirements of the patient. For example, if the concentrator can make 2.2 LPM of 90% O₂ and the patient only needs FGF of 6 LPM, then only 3.8 LPM of ambient air is needed for blending, providing 6 LPM of 47% O₂.

In spontaneous breathing mode, it is helpful for ease of use to provide a concentration of 40% O₂, since most adults require less than 8 LPM of FGF, and providing this concentration requires a concentrator capable of producing 2.2 LPM of 90% O2 which can be made relatively small (<10 lbs.).

The patient can breathe at any frequency and with any tidal volume in spontaneous mode.

The system can be optionally used in a monitoring mode whereby the ventilator and concentrator are not operative and only patient monitoring is active. The patient may be breathing spontaneously on the circuit with the air pump 27 providing FGF to the circuit.

The spontaneous breathing circuit consists of the bellows 36, the one-way valve 45 from the bellows to the inspiratory limb of the spontaneous cartridge, the cartridge (which contains the optional filter 55), an inspiratory hose 54 with a Y-piece 51 connected to a plastic oxygen mask 53 (without holes to prevent dilution), an expiratory hose 54a, a one-way expiration valve 50 in the cartridge, and a sequential rebreathing valve 52 in parallel to the one-way expiration valve 50.

In spontaneous breathing mode the system does not generally provide assisted ventilation, although it may in some instances. During inhalation gas in the inspiratory limb and bellows is pulled through the optional filter 55 in the cartridge and through the patient inspiratory tube 54 to the mask 53. The patient exhales through the expiratory tube and out to ambient through the one-way exhalation valve 50. The sequential rebreathing valve 52 triggers when the patient's continues to inspire once the bellows 36 is empty, which occurs in general when his breathing rate exceeds the rate of FGF into the circuit.

According to one embodiment of the invention, the portable respiratory device operates with battery, DC or AC power.

Optionally, the device may house up to two batteries, preferably lithium polymer due to energy density, mounted internally and accessible at the end of the device. Optionally, the device operates on a battery for approximately 1.25 hours (2.5 hours per set). While operating from AC, the device may optionally trickle charge internal batteries.

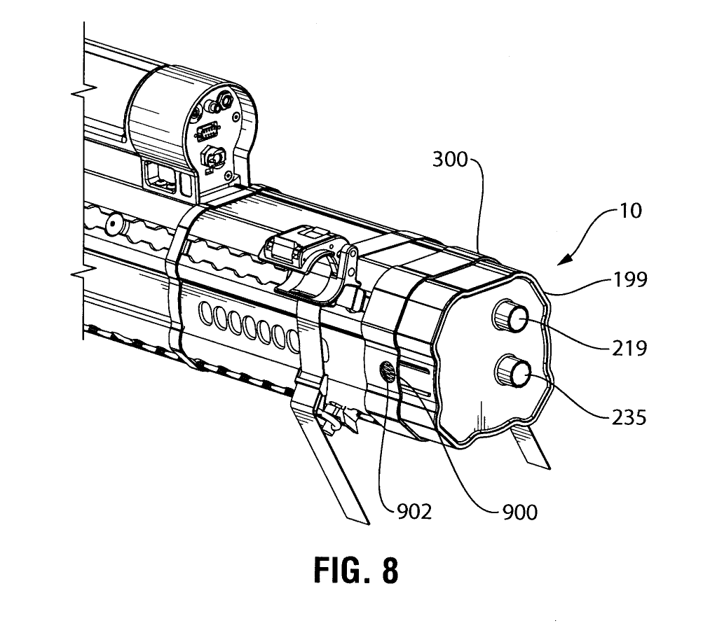

Reference is made to FIG. 8, which shows the cartridge 10, mounted in a seat 300. The cartridge 10 is configured to be used when the patient is being ventilated. The cartridge 10 is easily removable and replaceable with another cartridge for use when the patient is spontaneously breathing. The cartridge 10 includes a locking tab 900 on two opposing sides. The locking tab 900 engages an aperture 902 through the wall of the seat 300. To release the cartridge 10 from the seat 300, the user pushes inwardly on the locking tabs 900 to disengage them from the apertures 902. To engage the cartridge 10 in the seat 300, the cartridge 10 is simply pushed into the seat 300 until the locking tabs 900 lock in the apertures 902.

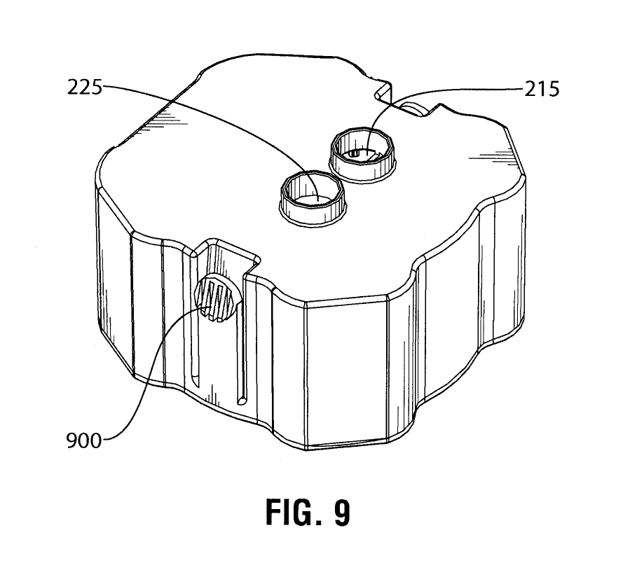

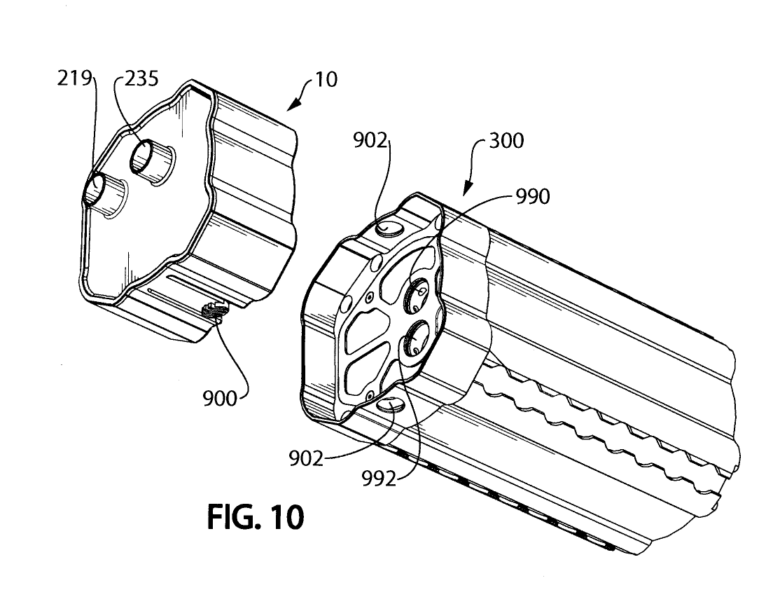

The cartridge 10 includes an inspiratory inlet 215 (FIG. 9), an inspiratory outlet 219 (FIG. 10), an expiratory inlet 235 (FIG. 10) and an expiratory outlet 225 (FIG. 9). When the cartridge 10 is seated, the inspiratory inlet 215 communicates with the one-way valve 45 at the inspiratory outlet, shown at 992, of the bellows 36, and the expiratory outlet communicates with the one-way valve 46 at the expiratory inlet, shown at 990, of the bellows 36.

Referring to FIG. 11, the cartridge 10 includes a CO2 scrubber 199 and a pass-through expiratory conduit 41. As shown in FIG. 1, air from conduit section 38 enters the scrubber 199 via the scrubber inlet port 215. The scrubber 199 includes a housing 205 and an internal structure 207 that together define a two tier helical airflow pathway. The housing 205 includes a first housing element 206a and a second housing element 206b, which are removably connectable together. A first end plate 210 and a second end plate 230 are positioned at opposing longitudinal ends of the scrubber 199 and are part of the first and second housing elements 206b and 206a respectively.

The internal structure 207 includes an internal divider 220 that is generally midway between the first and second end plates 210 and 230. The plates 210, 220 and 230 are all generally perpendicular to the longitudinal axis of the scrubber 199 and of the portable life support apparatus 1102.

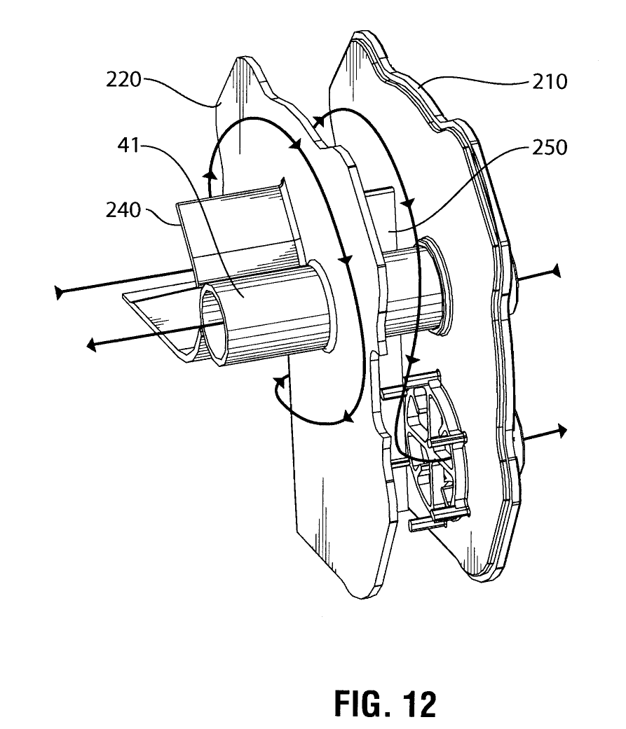

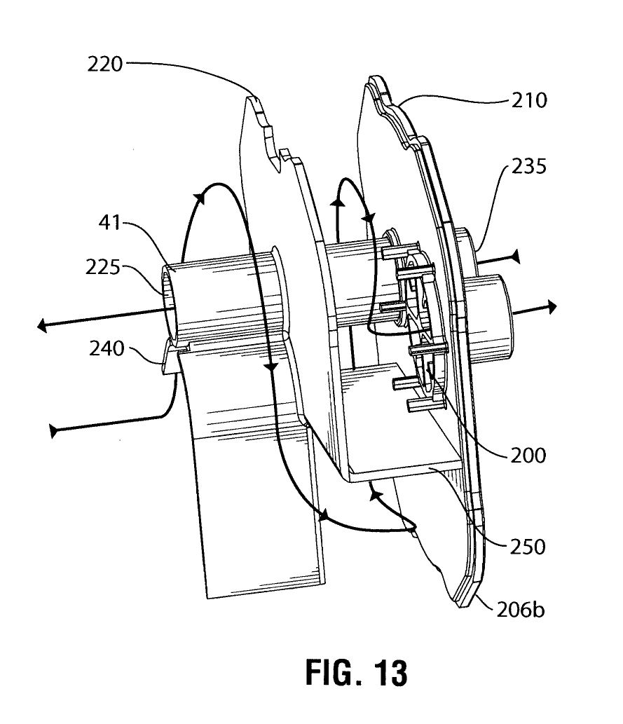

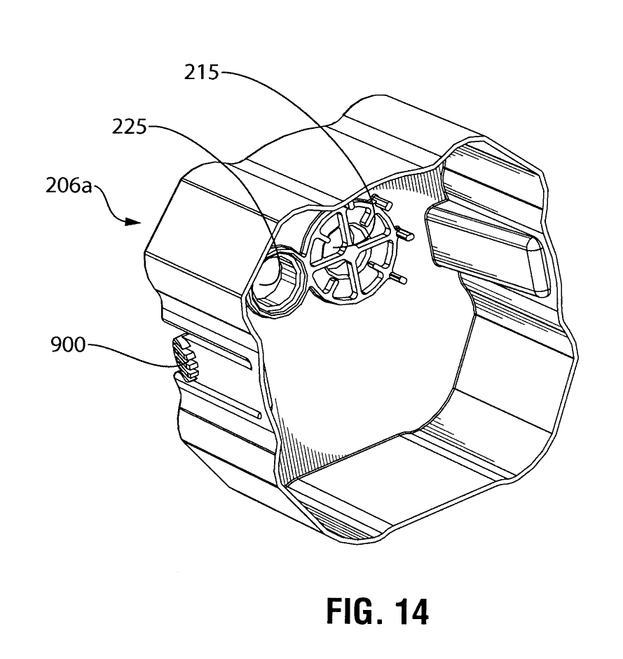

The internal structure 207 further includes wall structures 240 and 250 that cooperate with the plate 220 and with the housing 205 to urge the air along the aforementioned generally helical flow path. The interior wall structures 240 and 250 are better shown in FIGS. 12 and 13 and may be oriented substantially perpendicularly to surfaces 210, 220 and 230. FIG. 11 also shows scrubber outlet port 200 which is fluidly connected to patient inspiratory tube 39 (FIG. 1).

Scrubber material 217 is present through the inspiratory air flow path through the housing 205. Only a small portion of the total quantity of scrubber material 217 in the scrubber 199 is shown in FIG. 8. The scrubber material may be any suitable scrubber material, such as, for example soda lime.

Pass-through conduit 41 (also shown in FIG. 1) interconnects conduit sections 40 and 42 and passes right through the scrubber 199 so that air passes through the conduit 41 without contact with the scrubbing material, and includes scrubber expiratory inlet 235 leading from the y-piece (FIG. 1) via conduit section 42 (FIG. 1) and scrubber expiratory outlet 225.

The scrubber housing 205 and internal structure 207 are easily disassemblable for easy replacement of the scrubber material 217 and easy reassembly, as illustrated in the exploded view in FIGS. 11,12.

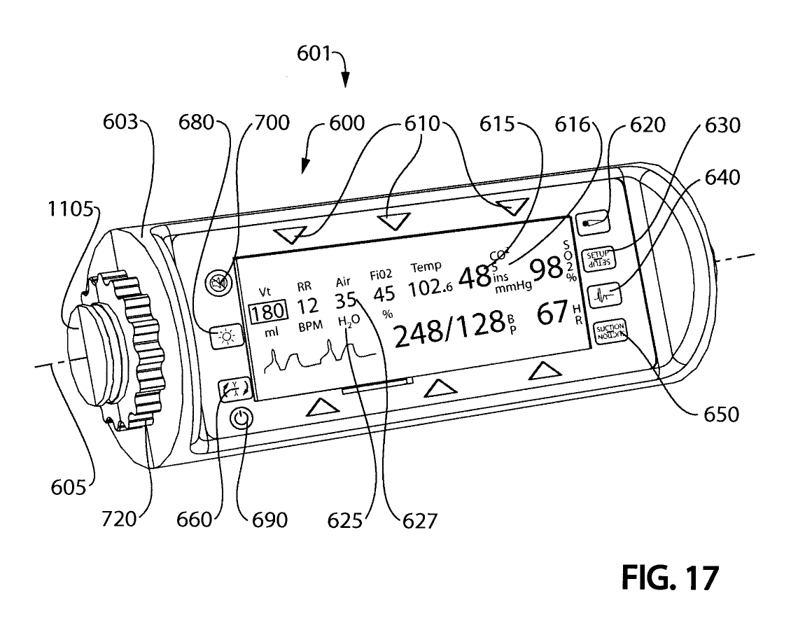

Referring to FIG. 17, the portable life support apparatus 1102 includes a display 600 for displaying patient vital statistics including; O₂ saturation, continuous or intermittent non-invasive blood pressure, CO₂, inspired O₂ concentration, ECG, heart rate, temperature, airway pressure, respiratory rate, and tidal volume. The display 600 may be a vacuum fluorescent display with adjustable brightness and a stealth mode. Additionally, the device 1102 may include one or more alarm lights 1150 (eg. four alarm lights 1150) positioned on the exterior. In embodiments wherein there is a plurality of the alarm lights 1150, the alarm lights 1150 may be dispersed about the perimeter of the device 1102 so as to increase the likelihood that one of them will be visible to a user, with less need to be concerned with the orientation of the device 1102.

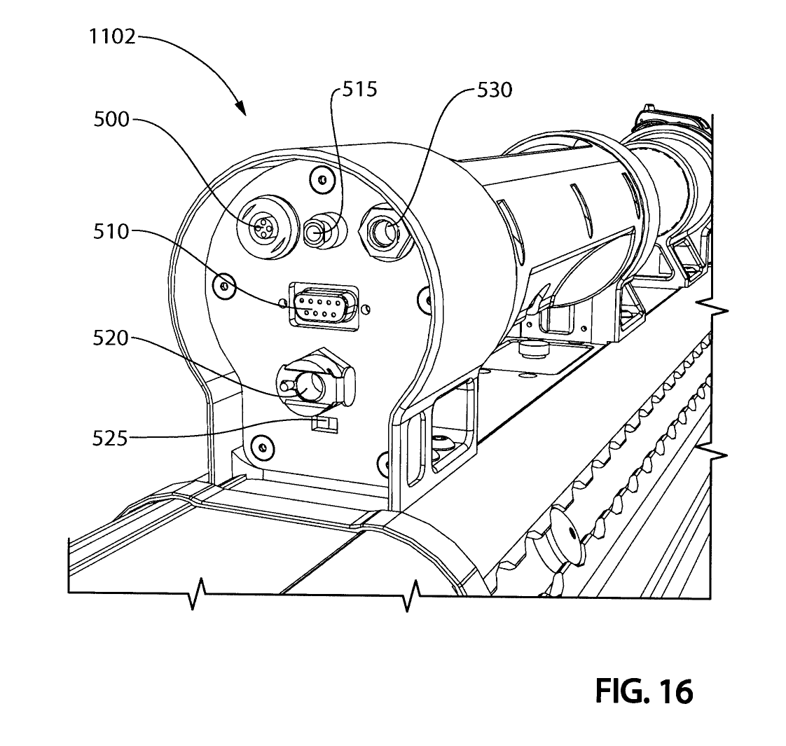

As shown in FIG. 16, the device 1102 may include input ports which may, for example, be located proximally to the display 600, which optionally houses a master control board for receiving data from an electrocardiogram (for example, data from a three lead ECG—port 500), an oxygen saturation meter (port 510), for example, a pulse oximeter (e.g. Nonin brand), a blood pressure cuff, either conventional—device has an air pump (not shown) and cuff inflation port 520) or acoustic (with extra microphones and noise cancellation features that are better adapted for helicopter noise cancellation—ports 520 and 525), gas sampling (sampling port from position proximal to patient—for example from Y piece 34 shown in FIG. 1—sampling port 515 leads to oxygen and CO2 monitors, optionally a rapid response infrared CO2 monitor) and temperature (port 530); and related parameters may be shown on the display 600, as shown in FIG. 17.

Persons skilled in the art will appreciate that a dedicated control board may be allocated for machine intelligence related to ventilator controls (tidal volume, airway pressure and BPM), concentrator control (oxygen concentration %, rate of operation), display controls etc.

As shown in FIG. 17, the screen 600 controls may set to display (to left to right) tidal volume in ml, BPM (breaths per minute), inspiratory airway pressure 625 in cm H₂0 in the inspiratory conduit, expiratory airway pressure 627 in cm H20, oxygen concentration, temperature, carbon dioxide concentration (both patient expiratory gas 615 and just upstream from the scrubber 199—smaller number 5 shown at 616 in FIG. 17), oxygen saturation; bottom left to right—graphic output e.g. ECG, blood pressure—systolic/diastolic and heart rate. Also shown are user interface controls—e.g. membrane switch keys—for activating BP (blood pressure) measurement 620, setup 630, suction 650, screen orientation reversal 660, ECG (three lead) 640, soft keys—610, screen dimming 680 (including night vision mode), power 690 and alarm silencing 700. Standard OEM sensing and monitoring modules are well known to those skilled in the art.

The display 600 may be part of a display assembly 601 that also includes a housing 603 which may be polymeric. The display assembly 601 may also include a filter suitable to screen output frequency to make the display night vision goggle compatible. The display 600 may utilize membrane switch keys above and below the screen, as exemplified above.

The housing 603 permits rotation of the display 600 about a display axis 605 for viewing over a range of angles by a user and can be flipped more than 180° to accommodate various mounting positions of the life support device 1002 in relation to the user.

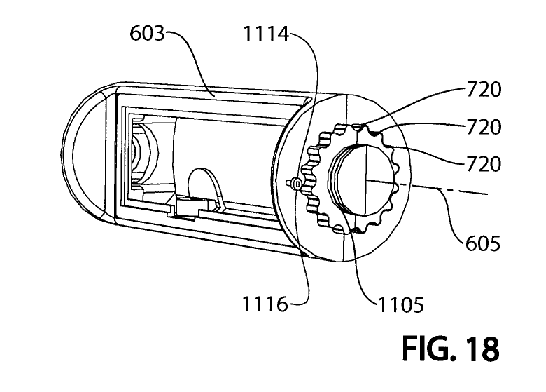

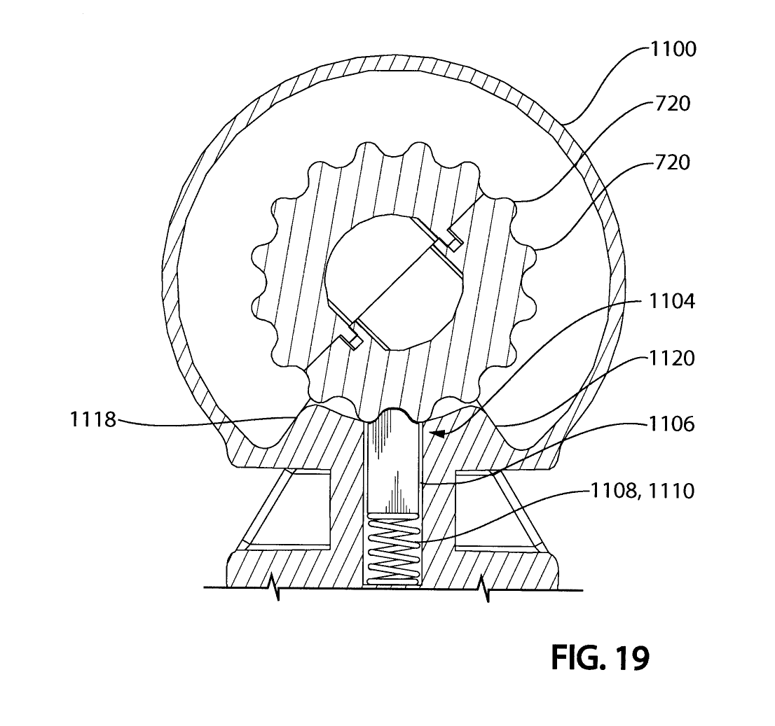

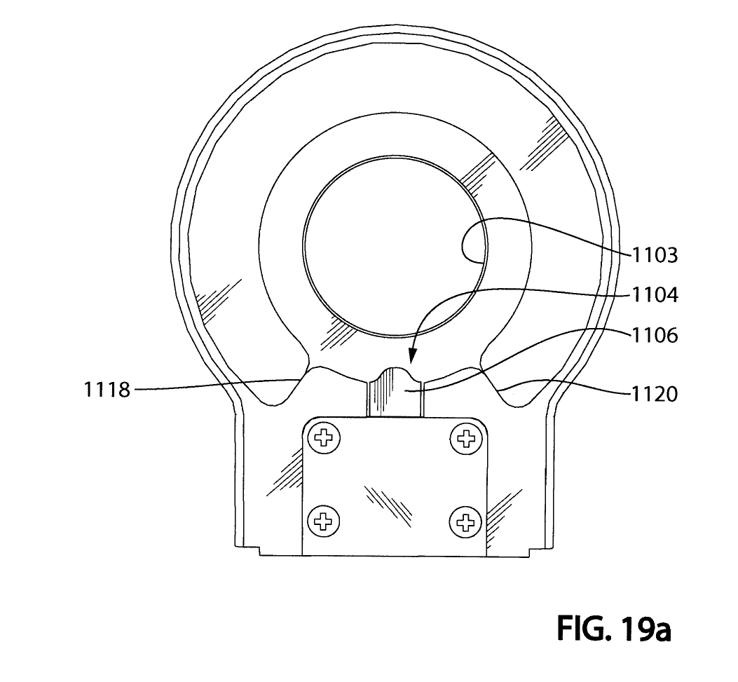

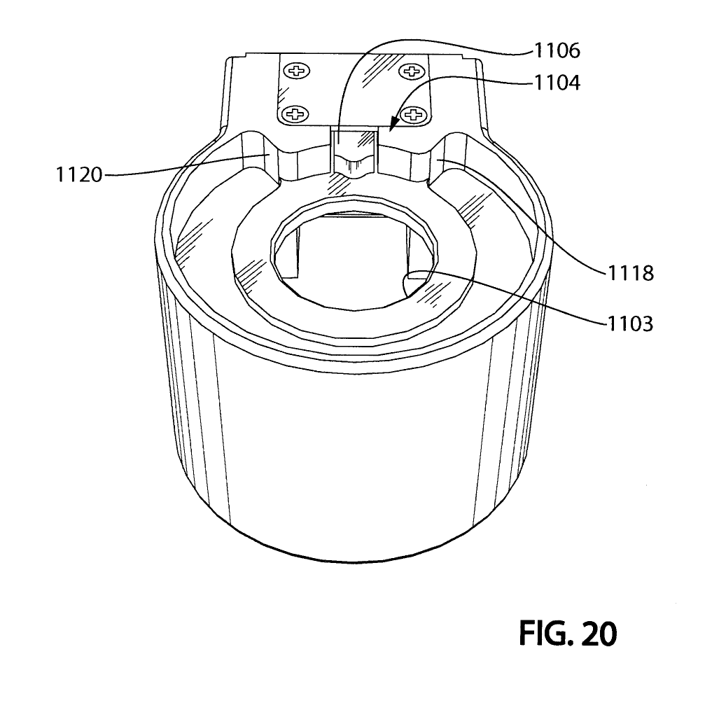

As shown in FIG. 15a, the housing 603 is rotatably supported in first and second end supports 1100 and 1102. Each end support 1100 and 1102 includes a bearing surface 1103 (see FIG. 19a) which supports a shaft portion 1105 (FIGS. 17 and 18) of the housing 603. Either or both of the end supports 1100 and 1102 may include a detent device 1104 (see FIG. 19) for engaging a plurality of teeth 720 positioned at one or both axial ends of the housing 603. The detent device 1104 includes a detent 1106 and a biasing member 1108, which may be, for example, a compression spring 1110. The detent device 1104 provides a selected amount of resistance to rotation for the display 600 so that the display 600 is less likely to rotate inadvertently when a user is entering input using the membrane switch keys described above. Additionally, the resistance to rotation provided by detent device 1104 is beneficial in that the display is less likely to inadvertently rotate as a result of vibration or other mechanical influence during use, for example, when the patient is being transported by helicopter to a medical facility.

Either or both axial ends of the display housing 603 (FIG. 18) may include a first limit surface 1114 and a second limit surface 1116 which engage corresponding limit surfaces 1118 and 1120 (FIG. 19a) in one or both of the end supports 1100 and 1102. The limit surface pairs, ie. 1114 and 1118, and 1116 and 1120, cooperate to limit the range of rotational travel of the display 600. The range of travel selected for the display 600 may be, for example, about 270 degrees.

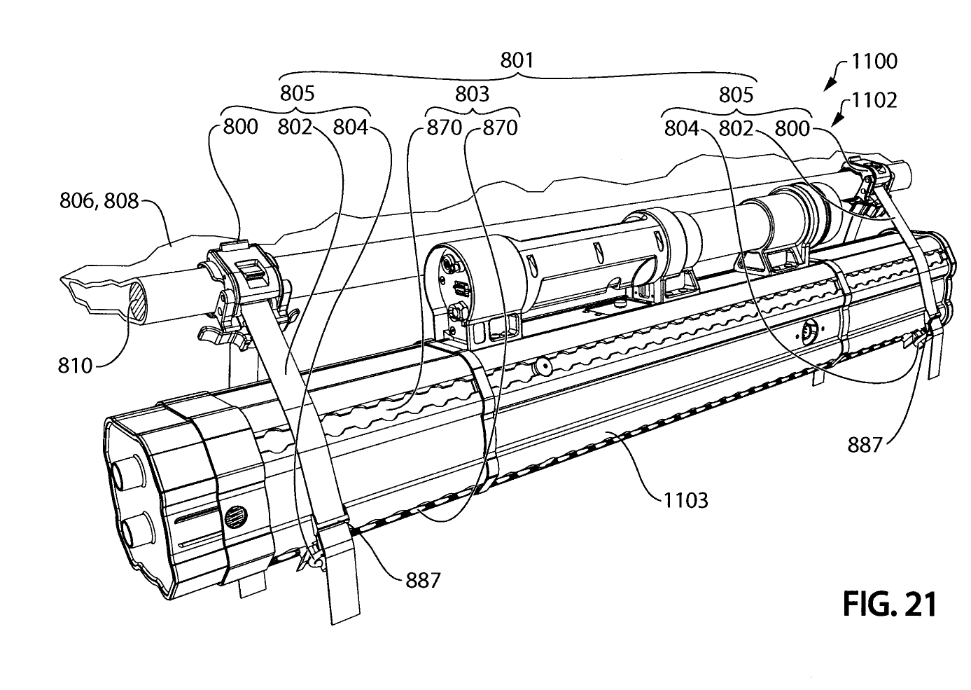

Reference is made to FIG. 21 which shows the components of the positioning system 801 in accordance with an embodiment of the present invention. The positioning system 801 may include one or more sets of: patient transport apparatus connectors 800, one or more straps 802, one or more life support system connectors 804 and a life support device interface 803. The life support device interface 803 may be common to all the sets. The patient transport apparatus connectors 800, one or more straps 802, one or more life support system connectors 804 make up a positioning structure 805.

The patient transport apparatus connector 800 connects to a patient transport apparatus 806, such as, for example, a stretcher and more particularly a NATO litter 808. The patient transport apparatus connector 800 may connect to the patient transport apparatus 806 in any suitable way for supporting the weight of the life support system 1100 (see FIG. 21). For example, the patient transport apparatus connector 800 may connect to one of the frame members, shown at 810, of the patient transport apparatus 806.

The patient transport apparatus connector 800 may mount releasably to the patient transport apparatus 806, and more particularly to the frame member 810. The patient transport apparatus connector 800 may include a clamp assembly 812 that is configured to clamp onto the frame member 810. The clamp assembly 812 includes a first clamp member 814 and a second clamp member 816, which cooperate with each other to clamp onto the frame member 810 (FIG. 21).

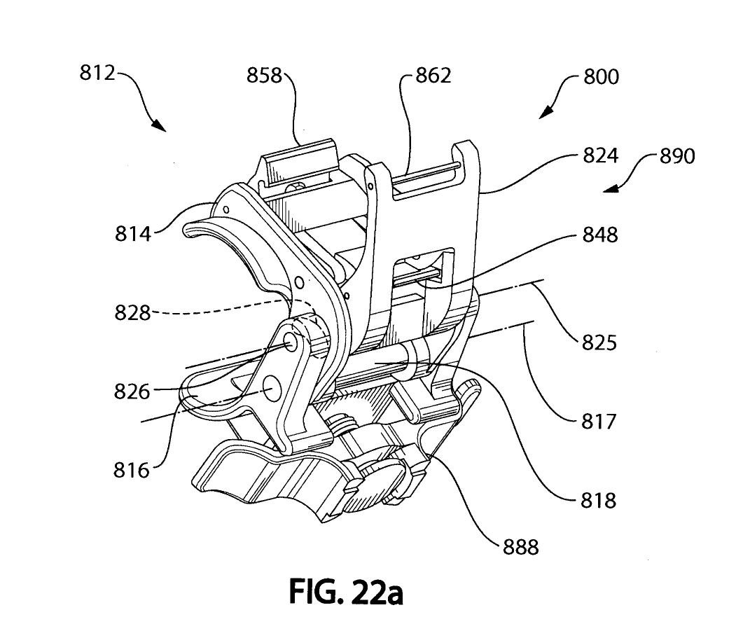

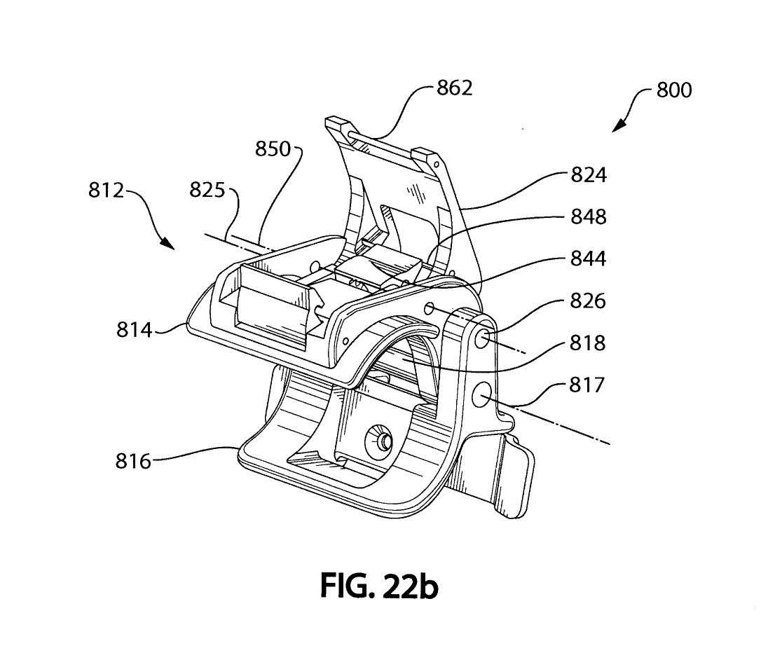

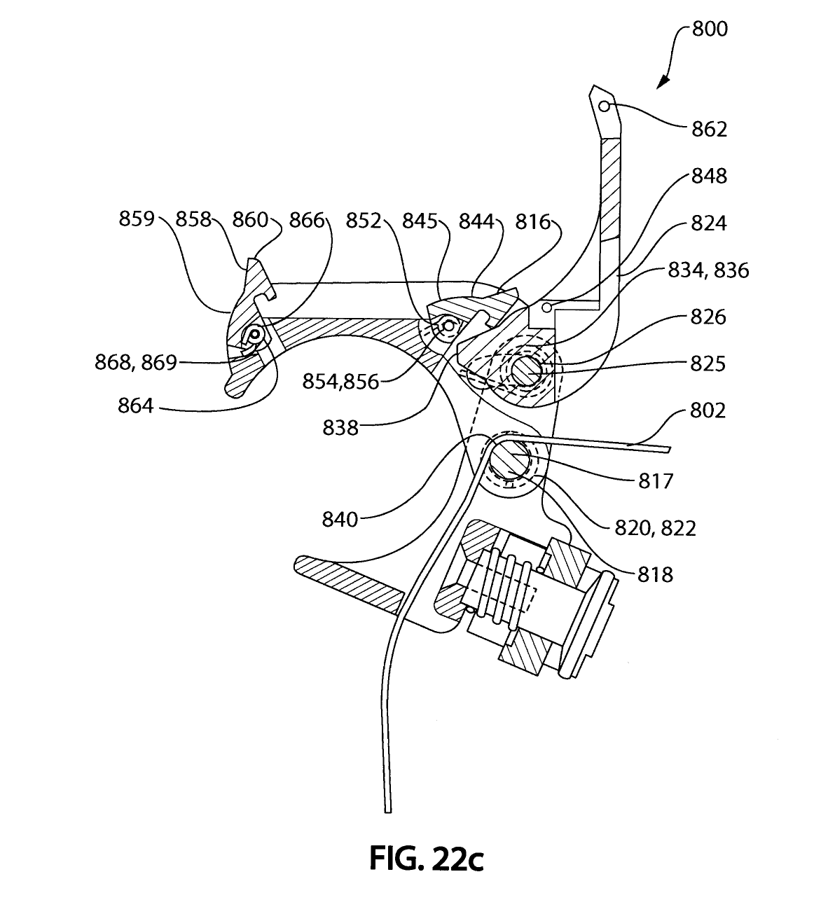

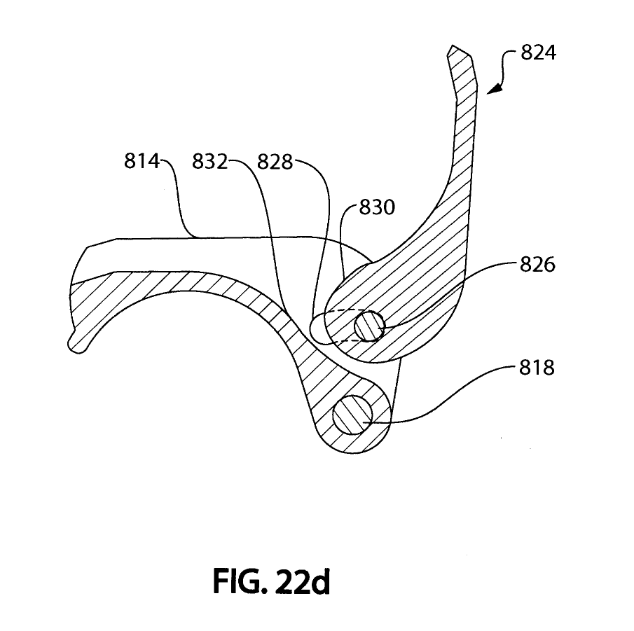

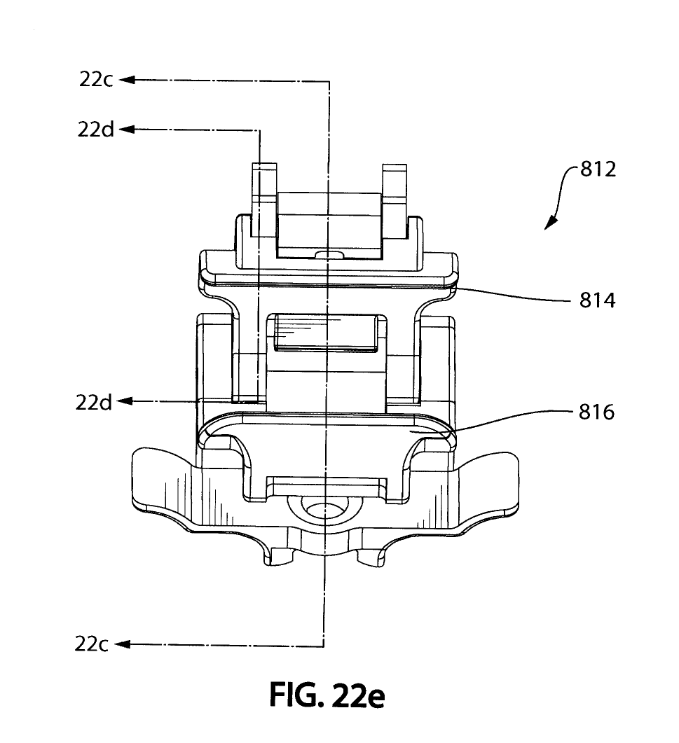

In the embodiment shown in FIG. 21, the patient transport apparatus frame member 810 is generally circular in cross-section and the first and second clamp members 814 and 816 (see FIG. 22a) are shaped in a suitable way to grip the circular shape. It will be understood that the cross-sectional shape of the patient transport apparatus frame member 810 may have any suitable shape, such as, for example, circular, elliptical, square or rectangular, for its function as a frame member, and it will be further understood that the clamp members 814 and 816 may have any suitable shape for gripping to the frame member 810.

It will further be understood that the clamp assembly 812 (FIG. 22a) need not only include two clamp members, but could include any suitable number of clamp members.

The first and second clamp members 814 and 816 may be pivotally connected to each other about a pivot axis 817. For example, a shaft 818 may extend through both clamp members 814 and 816, and may permit one or both of the clamp members 814 and 816 to rotate thereon.

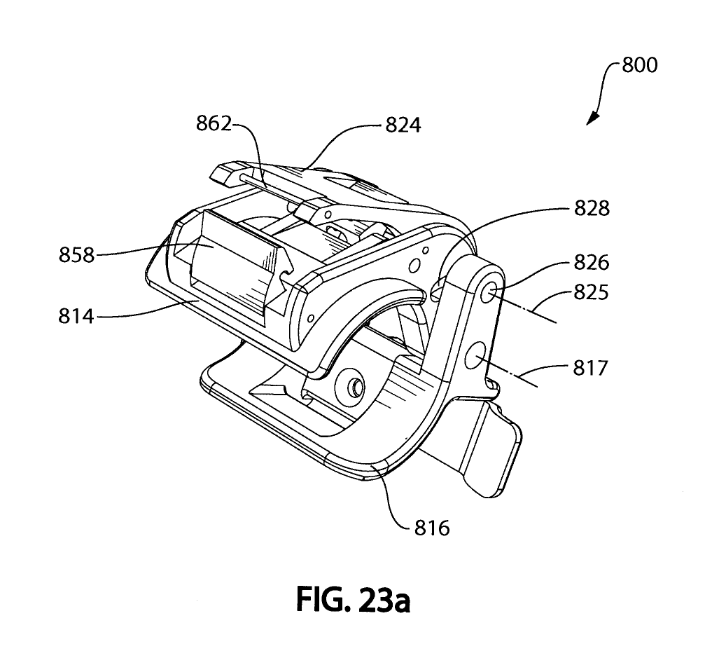

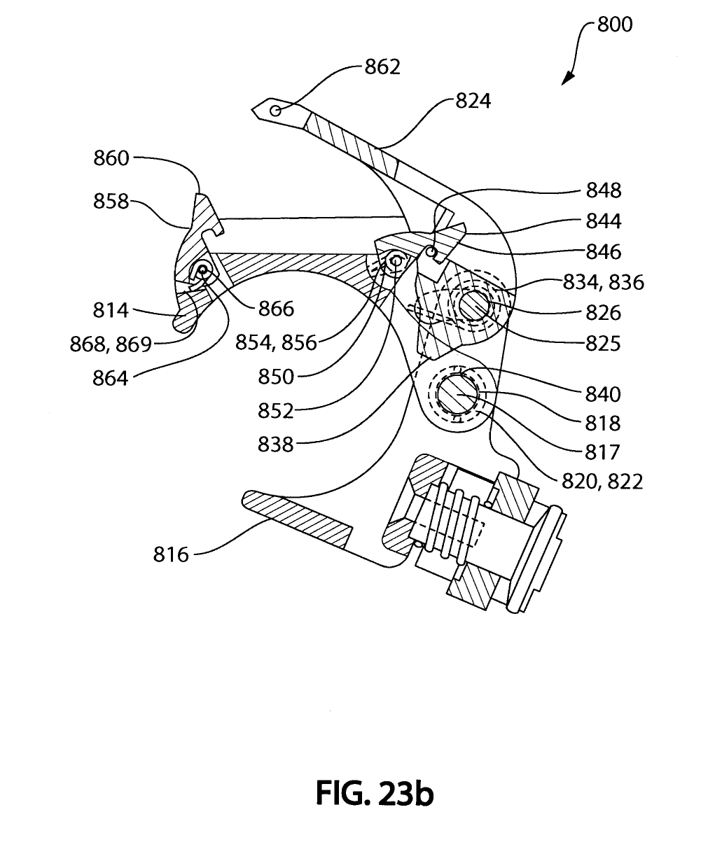

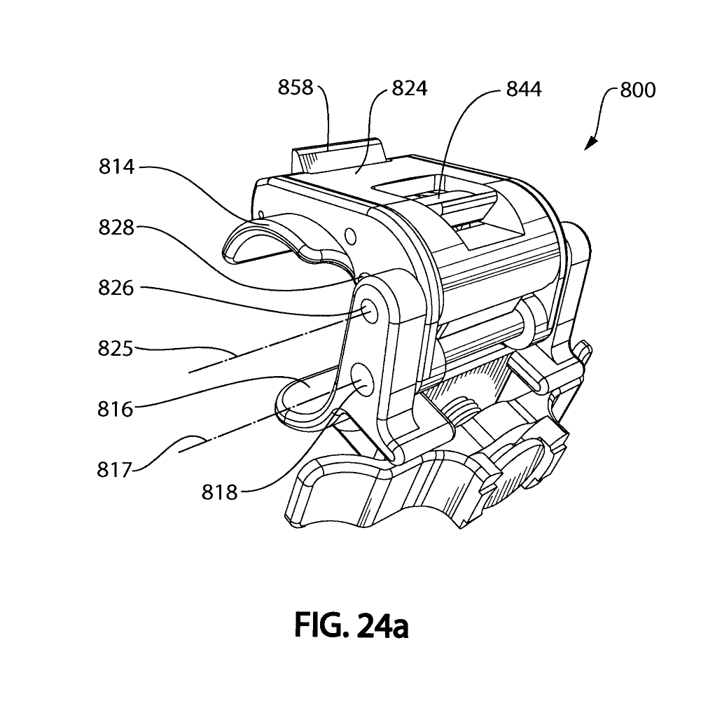

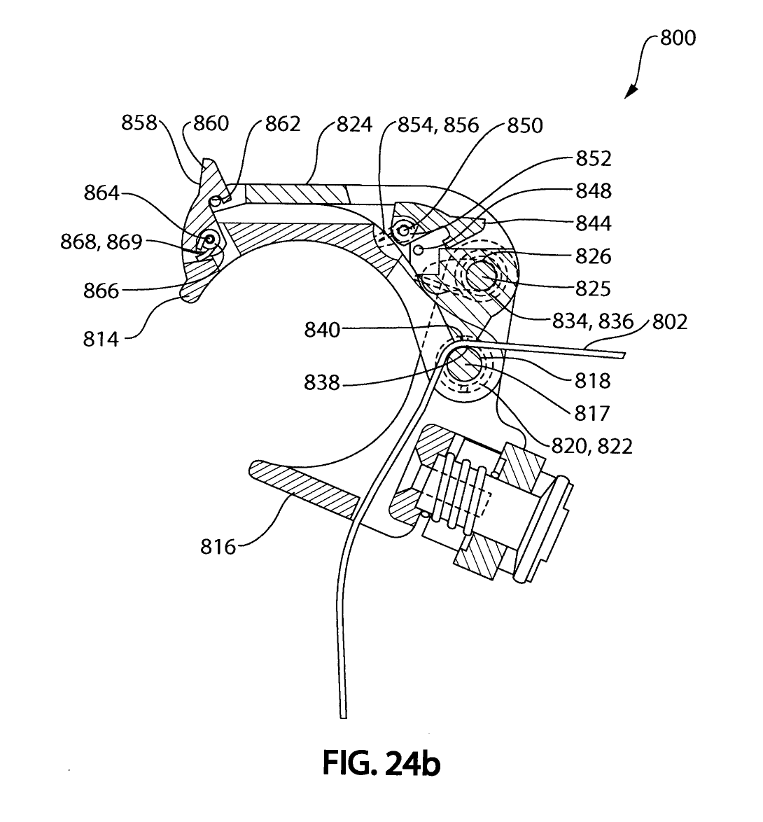

The patient transport apparatus connector 800 is movable between an open position, shown in FIGS. 22a, 22b, 22c, 22d and 22e, a patient transport apparatus-connected, strap-unlocked position, shown in FIGS. 23a and 23b, and a patient transport apparatus-connected, strap-locked position, shown in FIGS. 24a and 24b. The clamp assembly 812 includes a biasing member 820 (FIG. 22c), which biases the clamp members 814 and 816 apart, and thus biases the patient transport apparatus connector 800 towards its open position. The biasing member 820 may be any suitable biasing member, such as, for example, a spring, and more particularly a torsional spring, as shown at 822 in FIG. 22c.

The patient transport apparatus connector 800 further includes an actuation arm 824 that is used to urge the clamp members 814 and 816 towards each other. The actuation arm 824 is pivotally connected to the first clamp member 814 about a pivot axis 825. A shaft 826 passes through apertures in both the first and second clamp members 814 and 816 and the actuation arm 824. The aperture in the first clamp member 814 is shown at 828 (FIG. 22a) and is slotted, defining a path along an arc of a circle whose centre is the pivot axis 817 between the first and second clamp members 814 and 816, and whose function is explained further below.

Referring to FIG. 22d, the actuation arm 824 includes a clamp member driving cam surface 830, which is engageable with a receiving surface 832 on the first clamp member 814. As the actuation member 824 is rotated about the pivot axis 825 by a user, the clamp member driving cam surface 830 engages the receiving surface 832 (see FIGS. 22a and 22b) and drives the first clamp member 816 to pivot towards the second clamp member. Because the first clamp member 814 pivots only about the first and second clamp member pivot point 817, the aperture 830 is slotted to accommodate the pivoting movement of the first clamp member 814.

A biasing member 834 (FIG. 22c) biases the first clamp member 814 and the actuation arm 824 apart. The biasing member 834 may be any suitable biasing member, such as, for example, a spring, and more particularly a torsional spring, as shown at 836 in FIG. 22c.

Movement of the actuation arm 824 from the position shown in FIG. 22c to the position shown in FIG. 52b moves the first clamp member 814 to its closed position with respect to the second clamp member 816.

The actuation arm 824 further includes a first strap-locking surface 838 which can cooperate with a second strap-locking surface 840 that may be present, for example, on the shaft 818 (see FIG. 22c) to pinch the strap 802 and to therefore lock the patient transport apparatus connector 800 in place on the strap 802.

When the actuation arm 824 is in the position shown in FIG. 22c, the first and second strap-locking surfaces 838 and 840 are not engaged, and so the patient transport apparatus connector 800 is free to be slid along the strap 802. As noted above, the clamp members 814 and 816 are in their open position relative to each other.

When the actuation arm is in the position shown in FIG. 23b, the first and second strap-locking surfaces 838 and 840 may be closer together than they are when the connector 800 is position as shown in FIG. 22c, but they are not engaged, and so the patient transport apparatus connector 800 is free to be slid along the strap 842. The clamp members 814 and 816, however, are clamped onto the frame member 810 when in the position shown in FIG. 23b.

When the actuation arm is in the position shown in FIG. 22c, the first and second strap-locking surfaces 838 and 840 cooperate to pinch the strap 802 and therefore lock the patient transport apparatus connector 800 in place on the strap 802.

When the actuation arm 824 is in the position shown in FIG. 22c the biasing member 820 and the biasing member 834 hold the actuation arm 824 generally in the position shown. When it is desired for the actuation arm 824 to be in the position shown in FIG. 23b, a retaining member 844 can be employed to hold the actuation arm 824 in the position shown in FIG. 23b, against the biasing force of the biasing members 820 and 834.

The retaining member 844 may have any suitable structure for releasably holding the actuation arm 824 in place in the position shown in FIG. 23b. The retaining member 844 may be an arm 845 that extends from the first clamp member 814 and that includes a hook portion 846. The hook portion 846 cooperates with a hook-receiving element 848 on the actuation arm 824 to hold the actuation arm 824 in the position shown in FIG. 23b. It will be appreciated that the arm 845 could alternatively be positioned on the actuation arm 824 and the hook-receiving element 848 could be positioned on the first clamp member 814.

The arm 845 may be pivotally movable about a pivot axis 850. A shaft 852 may extend through the first clamp member 814 and the retaining member 844 along the pivot axis 850 to support such pivoting movement.

A biasing member 854 may be provided to bias the retaining member 844 to move in a direction towards hooking an object. The biasing member 854 may, for example, be a torsional spring 856 about the shaft 852.

During movement from the position shown in FIG. 22c to the position shown in FIG. 23b, the biasing member 854 biases the retaining member 844 against the actuation arm 824. The arm 824 moves so that the hook receiving portion sweeps slightly past the hook portion 846 and can then move back slightly along its path for capture by the hook portion 846.

When the actuation arm 824 is held in the position shown in FIG. 23b, it can be released from that position and returned to the position shown in FIG. 22c by moving the actuation arm 824 forward slightly along the path (ie. towards the position shown in FIG. 24b). Once the actuation arm 824 has moved out of the bight of the hook portion 846, the user can move and hold the retaining member 844 out of the path of the actuation arm 824 and the actuation arm 824 can be moved back towards the position shown in FIG. 22c.

Alternatively, a user could continue moving the actuation arm 824 past the position shown in FIG. 23b towards the position shown in FIG. 24b. A retaining member 858 can be employed to hold the actuation arm 824 in the position shown in FIG. 24b, against the biasing force of the biasing members 820 and 834.

The retaining member 858 may have any suitable structure for releasably holding the actuation arm 824 in place in the position shown in FIG. 22c. The retaining member 858 may be an arm 859 that extends from the first clamp member 814 and that includes a hook portion 860. The hook portion 860 cooperates with a hook-receiving element 862 on the actuation arm 824 to hold the actuation arm 824 in the position shown in FIG. 24b. It will be appreciated that the arm 859 could alternatively be positioned on the actuation arm 824 and the hook-receiving element 862 could be positioned on the first clamp member 814.

The arm 859 may be pivotally movable about a pivot axis 864. A shaft 866 may extend through the first clamp member 814 and the retaining member 858 along the pivot axis 864 to support such pivoting movement.

A biasing member 868 may be provided to bias the retaining member 858 to move in a direction towards hooking an object. The biasing member 868 may, for example, be a torsional spring 869 about the shaft 866.

During movement from the position shown in FIG. 23b to the position shown in FIG. 24b, the biasing member 868 biases the retaining member 858 against the actuation arm 824. The arm 824 moves so that the hook receiving portion sweeps slightly past the hook portion 860 and can then move back slightly along its path for capture by the hook portion 860.

Other structures may alternatively be employed to hold the actuation arm 824 at the positions shown in FIGS. 23b and 24b. For example, while the embodiment shown in FIGS. 22c, 23b and 24b has two separate hook receiving portions on the actuation arm 824, it is alternatively possible to have a single hook receiving portion that is engageable by either of two hooks for holding the actuation arm 824 in the two positions shown in FIGS. 23b and 24b.

When the actuation arm 824 is held in the position shown in FIG. 24b, it can be released from that position and returned to the position shown in FIG. 23b by moving the actuation arm 824 forward slightly along its path. Once the actuation arm 824 has moved out of the bight of the hook portion 860, the user can move and hold the retaining member 858 out of the path of the actuation arm 824 and the actuation arm 824 can be moved back towards the position shown in FIG. 23b, either manually, or optionally by the biasing member 834.

It will be understood that the patient transport apparatus connector 800 could alternatively be configured so that the actuation arm 824 drives the second clamp member 816 towards the first clamp member 814.

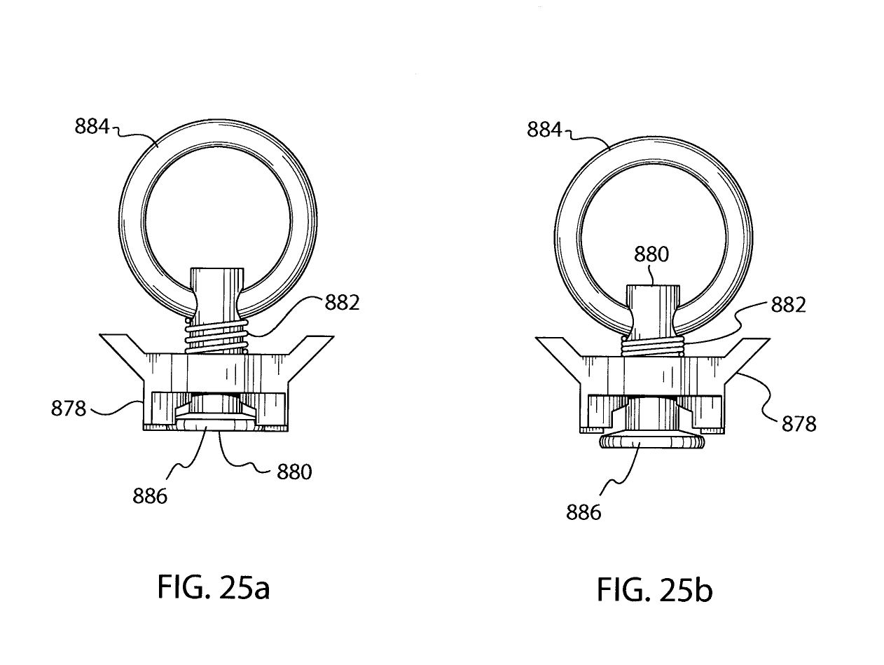

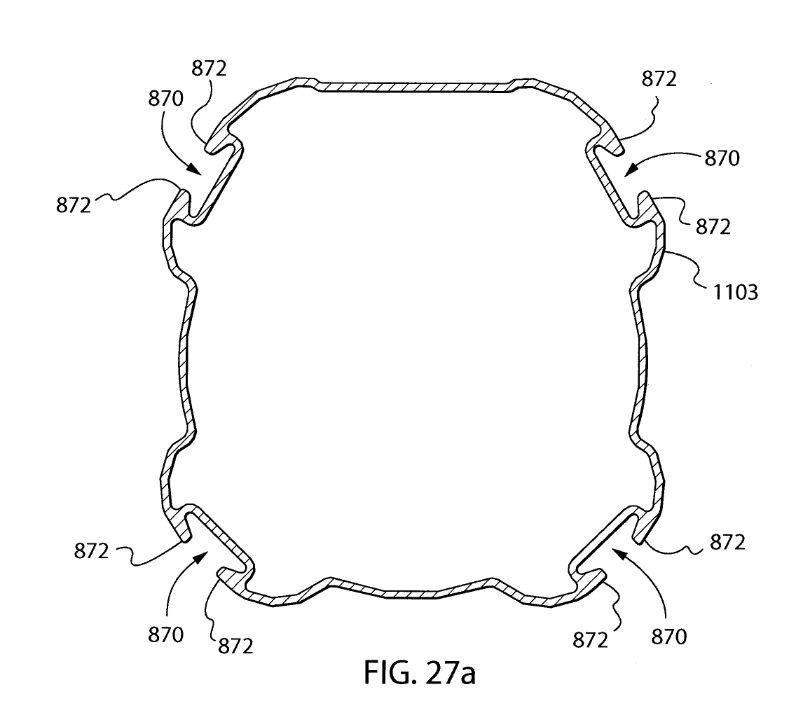

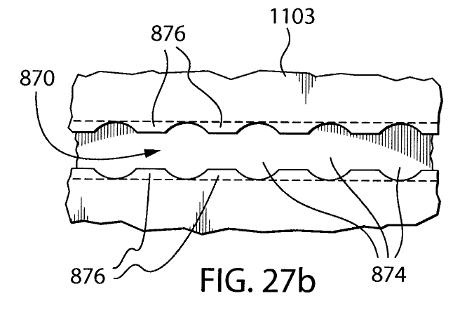

Reference is made to FIGS. 25a and 25b, which show the life support device connector 804 and which illustrate its operation. The life support device connector 804 may connect to the life support device in any suitable way. For example, the portable life support apparatus 1102 may include a exterior 1103 with at least one undercut channel 870 thereon, as shown in FIGS. 27a and 27b. As a result of the undercut, the channel 870 includes overhangs 872 on each side. As best shown in FIG. 27b, the overhangs 872 are cut away to form circular cutouts 874 (or any other suitable shape) having a selected spacing from one another. The individual overhang elements that are present between adjacent circular cutouts are shown at 876.

As shown in FIG. 27a, the exterior 1103 may include a plurality of channels 870 that extend axially. The channels 870 are spaced at regular intervals about the perimeter of the exterior 1103. The channels 870 make up the life support device interface 803 referred to above.

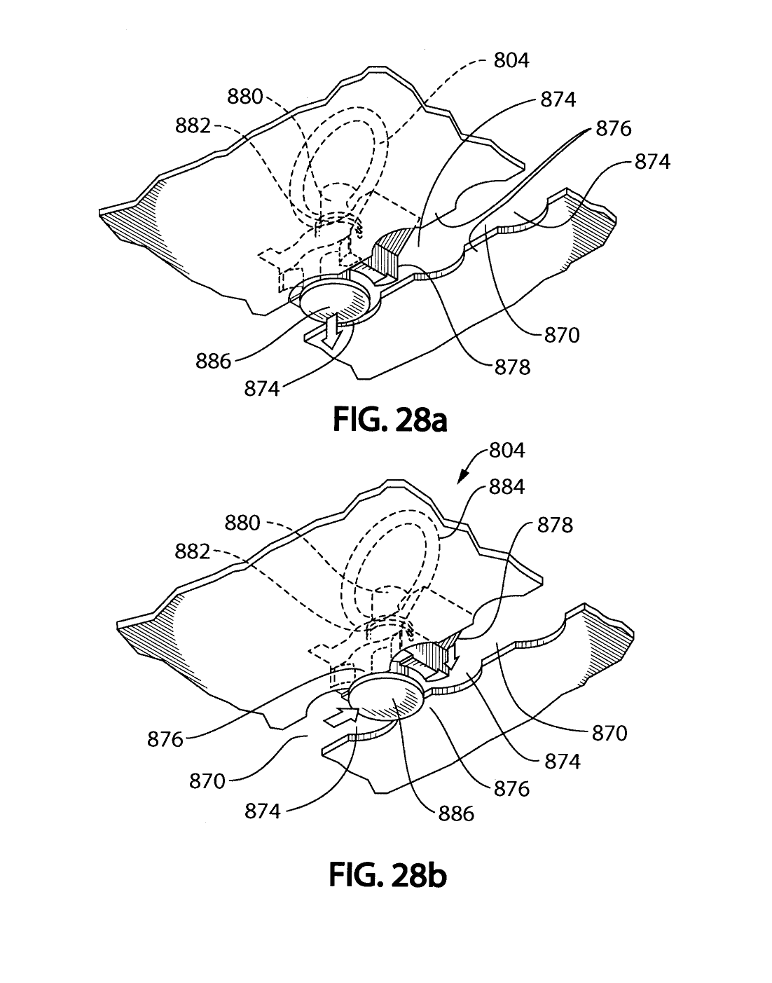

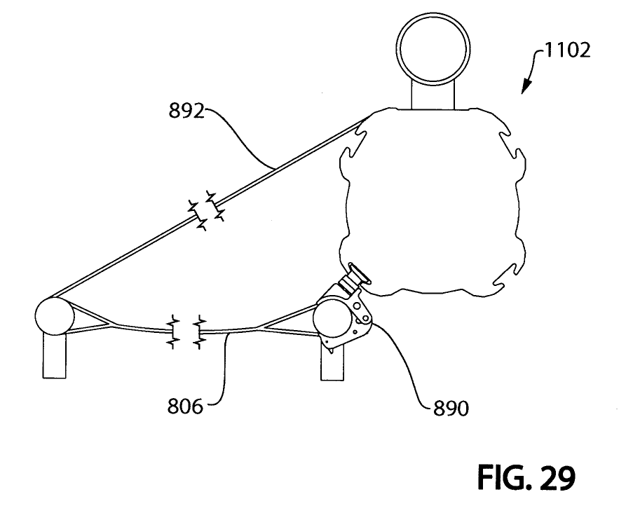

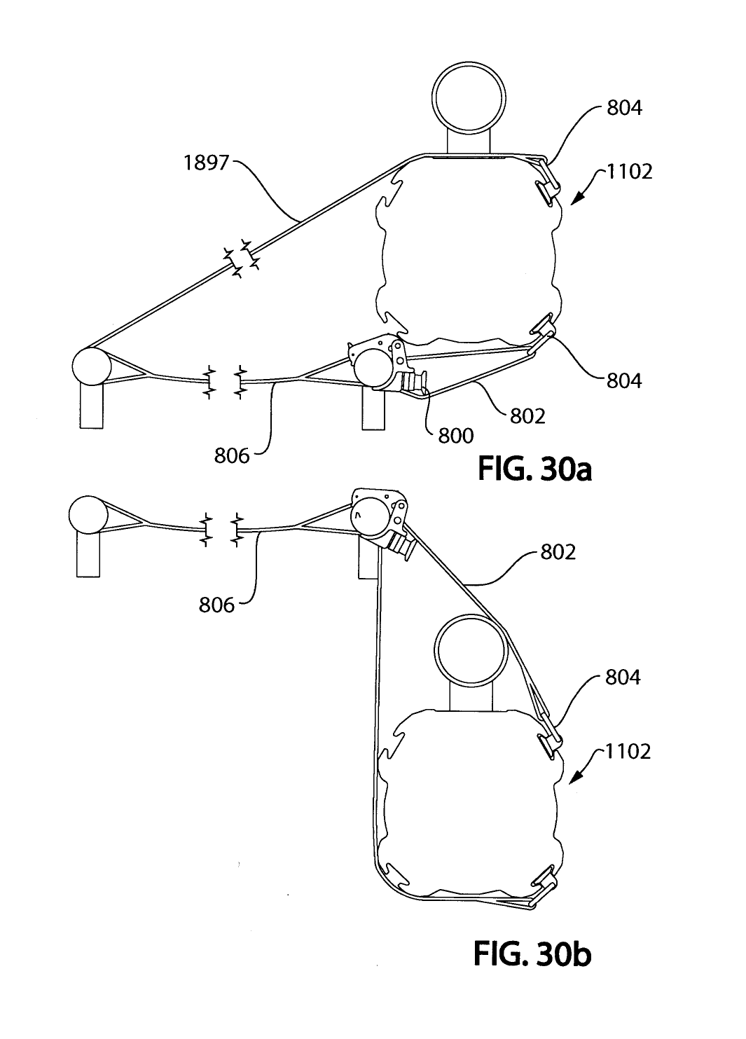

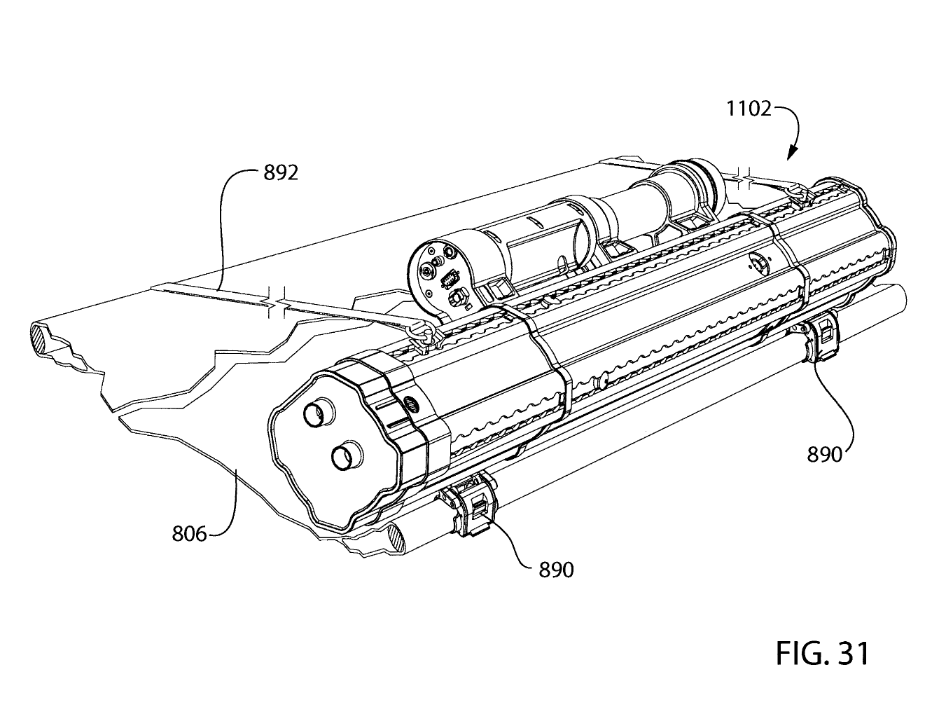

Referring to FIG. 54, the life support device connector 804 includes a body 878, a movable locking element 880, a biasing member 882 and a strap connector 884. The locking element 880 is movable relative to the body and has an enlarged head portion 886 which may be circular (or any other suitable shape). The locking element 880 is biased by the biasing member 882 to drive the head portion 886 towards the body 878. The biasing member 882 may be any suitable biasing member such as, for example, a coil compression spring. The head portion 886 of the locking element 880 is sized to fit within the circular cutout 874 in the channel 870 on the life support device exterior 1103 (see FIG. 28a). A user may push the locking element 880 downwards, against the biasing force of the biasing element, as shown in FIG. 25b.

To lock the life support device connector 804 on the exterior 1103, the body 878 is positioned to rest on a pair of adjacent overhang elements 876, so that the head portion 886 is aligned with a cutout 874 in the channel 870, as shown in FIG. 28a. The locking element 880 can be pushed inwards by a user, so that the head portion 886 enters the channel 870. By pushing the head portion 886 down sufficiently into the channel 870, the life support device connector 804 may be slid along the channel 874 such that the head portion 886 slides underneath the overhang elements 876. The life support device connector 804 may be slid to a position where the body 878 straddles an overhang element 876 (see FIG. 28b). The biasing member 882 urges the head portion 886 upwards towards the body 878 to clamp the overhang elements 876 that are straddled by the body 878 on either side of the channel 874.

Such a connector is sold by ANCRA (40340-27—Single Stud Track Fitting, and may be used with track 40467-33-144, which is the basis for the shape of the channel 870). Other suitable connectors may instead be used.

In the embodiment shown in FIG. 21, the positioning system 801 includes two sets of positioning structure 805, wherein each set includes two life support device connectors 804 which are mountable at spaced apart positions about the perimeter of the portable life support apparatus 1102, a patient transport apparatus connector 800 that is connectable to a patient transport apparatus 806, and a strap 802 that extends between the two life support device connectors 804 and through the patient transport apparatus connector 800.

Referring to FIG. 21, the strap 802 may be adjustable in length. For that purpose, the strap may include any suitable length adjustment mechanism 887, such as, for example, a length adjustment buckle.

Referring to FIG. 21, the positioning system 801 permits the portable life support apparatus 1102 to be positioned in a plurality of positions relative to a patient transport apparatus 806. For example, by extending the strap 802, the portable life support apparatus 1102 may hang from the patient transport apparatus 806. By adjusting the position of the life support device connectors 804 about the perimeter of the portable life support apparatus 1102, and/or by adjusting the position of the patient transport apparatus connector 800 along the strap 802, the orientation of the portable life support apparatus 1102 can be controlled.

The portable life support apparatus 1102 could, for example, be positioned on the patient transport apparatus 806 optionally with the air of an extra strap 897 (FIG. 30a) for entry into a patient transport vehicle, such as a helicopter, and can then be repositioned in a hanging position (FIG. 30b).

By shortening the straps 802, the portable life support apparatus 1102 can be brought into relative close proximity to the patient transport apparatus connector 800, which reduces the magnitude of any swinging that might take place during use. It is possible that the straps 802 can be shortened sufficiently to bring the portable life support apparatus 1102 into contact with the patient transport apparatus connector 800, which can effectively create a generally rigid connection between the portable life support apparatus 1102 and the patient transport apparatus 806.

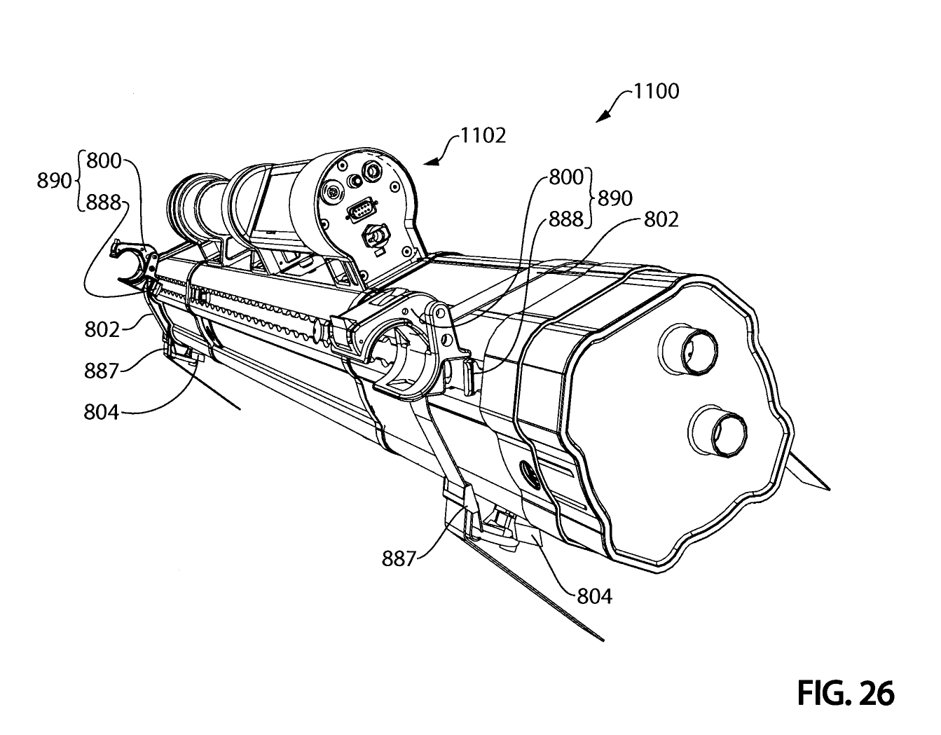

Referring to FIG. 22a, a life support device connector 888, which may be similar to the connector 804, is optionally integrally joined with the patient transport apparatus connector 800 to form a rigid patient transport apparatus/life support device connector 890 which permits the rigid connection directly between the patient transport apparatus 806 and the portable life support apparatus 1102 (see FIG. 26).

Referring to FIG. 29, additional straps 892 may be employed to help hold the portable life support apparatus 1102 in a relatively fixed position relative to the patient transport apparatus 806 in cooperation with the rigid patient transport apparatus/life support device connector 890. It will be also be appreciated that the portable life support apparatus may be suspended above and below a side frame member of a portable patient transport apparatus using the clamps without the aid of any straps by using a track proximal to the bottom and top of the apparatus respectively. When suspended below the side frame member, the weight of the device rotates the device partially under the side frame member towards the center the portable patient transport apparatus.

The portable life support apparatus 1102 may include several control boards (eg. five control boards) in the life support apparatus, in addition to any boards that control patient monitors. The control boards are described as follows:

User Interface and main bus control—manages display screen and user buttons, communicates with other boards to manage traffic. It sends signals to other boards as to what to do and gets reports back, displays these on screen, handles alarm conditions and warning lights.

Main Power—controls battery vs wall power, switching between batteries when discharged, monitoring power etc., including measuring battery temperature for overheating

O₂ Controller—controls the oxygen concentrator. Controls MiniOx sensor for measuring O₂ in the bellows as well as sieve pressure sensors used for controlling concentrator valves. Controls the valves on the concentrator. It has a motor controller for controlling the concentrator pump motor. It coordinates with the ventilator control to ensure it does not provide oxygen enriched air to the circuit at the end of inspiration, because at that point it's impossible to correct the delivered volume for this amount.

Ventilator Control—controls the blower motor to provide the required volume and breath frequency, as well as PEEP. This also contains and airway pressure sensor and the bellows position sensor. It also contains a differential pressure sensor which can be used for a flow sensor to measure flow at the patient's mouth to measure what actually got delivered, as opposed the measuring bellows displacement. Differential pressure divided by the known circuit resistance gives flow). This board controls the mixing pump and measures delivered volume using an estimate of O2 enriched air volume from the concentrator.

Sensor Control Board—controls all of the off the shelf patient monitoring devices, some of which have their own boards. Contains/controls the patient CO₂ and O₂ sensors and their sampling pump, pressure sensor for measuring pressure in the sampling line (since this affects the reading of the CO2 and O2 sensors and is preferably corrected for, and it also detects occlusion of the sampling line), contains a barometric pressure sensor for altitude corrections for the sensors. Additionally, knowing the altitude enables “tuning” the concentrator to work at different working pressures based on the altitude (i.e. the set of working pressures that optimizes the concentrator at sea level may not be the same set that optimizes at 8,000 ft.). This board also measures temperature in the housing as well.

Patient Monitors—CO₂ and O₂ as above (continuous waveform, calculates inspired and end-tidal from waveform), O₂ saturation and plethysmography (from pulse ox), heart rate (from either pulse ox or ECG or blood pressure cuff), non-invasive blood pressure (NIBP, both acoustic noise cancelling and oscillometric), ECG 3 lead, temperature, airway pressure (see above).

Some of the alarm conditions include:

Patient parameter out of present range, (eg. O2, CO2, SpO2, HR, BP, T, Airway pressure), system error (occlusion, leak, O2 low, CO2 high, battery low, valve failure, pump failure, ventilator failure and patient monitor failure).

The device 1102 may further have sufficient controls to operate in selected failsafe modes. For example, the device 1102 may be configured to operate if there is an O2 failure, in a ‘limpalong’ mode whereby the concentrator is not capable of producing O2 at 85% concentration. It may also ventilate using ambient air. In the event of a ventilator failure, an Ambu bag may be interposed in the breathing circuit for manual ventilation, using the oxygen concentrator and/or mixing pump as its supply.

The device 1102 includes optional alarm lights 1150 which may be visible along nearly 180 degrees of viewing angle. The lights may be red to indicate an urgent problem, yellow to indicate a problem that does not require urgent attention, green to indicate that everything is operating within selected ranges and infra-red when operating in stealth mode.

The device 1102 may be mounted along the edge of a stretcher or other patient transport device using the positioning system facing either direction, so that preferably the patient connections and tubes are closest to the head. The screen may be rotated and its contents flipped to make reading easier.

While the above description constitutes the preferred embodiments, it will be appreciated that the present invention is susceptible to modification and change without departing from the fair meaning of the accompanying claims.

1. A portable life support device comprising:
at least one ambient gas inlet;
a conditioned gas outlet;
a gas conduit assembly fluidly connected between the at least one ambient gas inlet and the conditioned gas outlet;
an oxygen concentrator fluidly connected to said gas conduit assembly;
a ventilator fluidly connected to said gas conduit assembly; and wherein said gas conduit assembly comprises at least one conduit section positioned upstream of said conditioned gas outlet for receiving oxygenated air from said oxygen concentrator and gas from the ventilator.

2. The portable life support device of claim 1, said ventilator includes an inspiratory reservoir and a controller for controlling the tidal volume of an inspiratory cycle and wherein said gas conduit assembly comprises at least one conduit section positioned upstream of said conditioned gas outlet for receiving oxygenated air from said oxygen concentrator and gas from the inspiratory reservoir.

3. The portable life support device of claim 2, wherein said conduit assembly comprises conduit sections of a re-breathing circuit including at least one expiratory conduit section for receiving exhaled air from a subject.

4. The portable life support device of claim 2, wherein said expiratory conduit section is fluidly connected to said inspiratory reservoir.

5. The portable life support device of claim 1, wherein said ventilator includes an inspiratory reservoir contractor and wherein said inspiratory reservoir is contractible.

6. The portable life support device of claim 1, wherein said inspiratory reservoir is the interior of a bellows and said inspiratory reservoir contractor is a blower and wherein said bellows comprises an exterior face for receiving a bellows-contracting air flow from said blower.

7. The portable life support device of claim 1 or 6, comprising a second ambient air inlet in fluid communication with said blower and wherein the blower propels ambient air entering said second ambient air inlet against the bellows.

8. The portable life support device of claim 7, wherein said bellows includes a PEEP valve and wherein gas exiting the PEEP valve exits the second ambient air inlet via the blower.

9. The portable life support device of claim 1, comprising a carbon dioxide scrubber.

10. The portable life support device of claim 2, wherein the oxygen concentrator, inspiratory reservoir and conduit sections of the re-breathing circuit are arranged substantially end to end in a longitudinal profile.

11. The portable life support device of claim 10, wherein the oxygen concentrator includes two sieve beds, and wherein and two sieve beds are arranged side by side and occupy longitiudinally similar positions in a first longitudinal segment of the apparatus.

12. The portable life support device of claim 11, wherein the inspiratory reservoir is located a second longitudinal segment of the apparatus located adjacent to the first longitudinal segment of the apparatus.

13. The portable life support device of claim 9, wherein the scrubber is included in a third longitudinal segment of the apparatus located adjacent to the second longitudinal segment of the apparatus.

14. The portable life support device of claim 13, wherein the device comprises interchangeable third longitudinal segments of the apparatus that are respectively releasably attached to the second longitudinal segment of the apparatus and are individually adapted to operate in respective ventilatory and spontaneous breathing modes of operating the apparatus.

15. The portable life support device of claim 14, wherein the device includes at least one detector that identifies operative attachment of the apparatus to at least one of the interchangeable third longitudinal segments of the apparatus.

16. The portable life support device of claim 9 or 14, wherein the apparatus is adapted to operate in a ventilatory mode and wherein the apparatus includes a releasably attached scrubber cartridge.

17. The portable life support device of claim 9 or 14, wherein the apparatus is adapted to operate in a spontaneous breathing mode and wherein the apparatus includes a releasably attached cartridge comprising a conduit assembly section that contains a one way valve out to atmosphere and an inspiratory relief valve.

18. The portable life support device of claim 17, wherein the cartridge is attached to a patient air way interface that does not have an expiratory outlet and wherein the conduit assembly further includes of a section of conduit of sufficient volume to store expired gas for re-breathing during inspiratory relief.

19. The portable life support device of claim 1, comprising at least one controller for processing data pertaining to respiratory parameters and a readable output display and wherein the readable output display is operatively connected to the at least one controller for displaying the parameters thereon.

20. The portable life support device of claim 1, wherein said readable output display is operatively attached to the second longitudinal section of the apparatus.

21-100. (canceled)
